(19) United States
(12) Patent Application Publication
(10) Pub. No.: US 2009/0142279 A1
(43) Pub. Date: Jun. 4, 2009

(54) NOVEL M3 MUSCARINIC ACETYLCHOLINE RECEPTOR ANTAGONISTS

(76) Inventor: Brian W. Budzik, Collegeville,, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/719,335

(22) PCT Filed: Nov. 15, 2005
(86) PCT No.: PCT/US05/41230

§ 371 Date: Feb. 17, 2009

Related U.S. Application Data
(60) Provisional application No. 60/627,822, filed on Nov. 15, 2004.

(51) Int. Cl. A61K 31/497; C07D 403/02

(52) U.S. Cl. 424/45; 544/357; 544/360; 544/230; 514/252.13

(57) ABSTRACT

Muscarinic Acetylcholine receptor antagonists and methods of using them are provided.

### FIELD OF THE INVENTION

This invention relates to novel derivatives of biaryl amines, pharmaceutical compositions, processes for their preparation, and use thereof in treating M₃ muscarinic acetylcholine receptor mediated diseases.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M1-M5, and each is the product of a distinct gene. Each of these five subtypes displays unique pharmacological properties. Muscarinic acetylcholine receptors are widely distributed in vertebrate organs where they mediate many of the vital functions. Muscarinic receptors can mediate both inhibitory and excitatory actions. For example, in smooth muscle found in the airways, M3 mAChRs mediate contractile responses. For review, please see Caulfield (1993 Pharmac. Ther. 58:319-79).

In the lungs, mAChRs have been localized to smooth muscle in the trachea and bronchi, the submucosal glands, and the parasympathetic ganglia. Muscarinic receptor density is greatest in parasympathetic ganglia and then decreases in density from the submucosal glands to tracheal and then bronchial smooth muscle. Muscarinic receptors are nearly absent from the alveoli. For review of mAChR expression and function in the lungs, please see Fryer and Jacoby (1998 Am J Respir Crit. Care Med 158(5, pt 3) S 154-60).

Three subtypes of mAChRs have been identified as important in the lungs, M1, M2 and M3 mAChRs. The M3 mAChRs, located on airway smooth muscle, mediate muscle contraction. Stimulation of M3 mAChRs activates the enzyme phospholipase C via binding of the stimulatory G protein Gq/11 (Gs), leading to liberation of phosphatidyl inositol-4,5-bisphosphate, resulting in phosphorylation of contractile proteins. M3 mAChRs are also found on pulmonary submucosal glands. Stimulation of this population of M3 mAChRs results in mucus secretion.

M2 mAChRs make up approximately 50-80% of the cholinergic receptor population on airway smooth muscles. Although the precise function is still unknown, they inhibit catecholaminergic relaxation of airway smooth muscle via inhibition of cAMP generation. Neuronal M2 mAChRs are located on postganglionic parasympathetic nerves. Under normal physiologic conditions, neuronal M2 mAChRs provide tight control of acetylcholine release from parasympathetic nerves. Inhibitory M2 mAChRs have also been demonstrated on sympathetic nerves in the lungs of some species. These receptors inhibit release of noradrenaline, thus decreasing sympathetic input to the lungs.

M1 mAChRs are found in the pulmonary parasympathetic ganglia where they function to enhance neurotransmission. These receptors have also been localized to the peripheral lung parenchyma, however their function in the parenchyma is unknown.

Muscarinic acetylcholine receptor dysfunction in the lungs has been noted in a variety of different pathophysiological states. In particular, in asthma and chronic obstructive pulmonary disease (COPD), inflammatory conditions lead to loss of inhibitory M2 muscarinic acetylcholine autoreceptor function on parasympathetic nerves supplying the pulmonary smooth muscle, causing increased acetylcholine release following vagal nerve stimulation (Fryer et al. 1999 Life Sci 64 (6-7) 449-55). This mAChR dysfunction results in airway hyperreactivity and hyperresponsiveness mediated by increased stimulation of M3 mAChRs. Thus the identification of potent mAChR antagonists would be useful as therapeutics in these mAChR-mediated disease states.

COPD is an imprecise term that encompasses a variety of progressive health problems including chronic bronchitis, chronic bronchiolitis and emphysema, and it is a major cause of mortality and morbidity in the world. Smoking is the major risk factor for the development of COPD; nearly 50 million people in the U.S. alone smoke cigarettes, and an estimated 3,000 people take up the habit daily. As a result, COPD is expected to rank among the top five as a world-wide health burden by the year 2020. Inhaled anti-cholinergic therapy is currently considered the “gold standard” as first line therapy for COPD (Pauwels et al. 2001 Am. J. Respir. Crit. Care Med. 163:1256-1276).

Despite the large body of evidence supporting the use of anti-cholinergic therapy for the treatment of airway hyperreactive diseases, relatively few anti-cholinergic compounds are available for use in the clinic for pulmonary indications. More specifically, in United States, Ipratropium Bromide (Atrovent©; and Combivent©, in combination with albuterol) is currently the only inhaled anti-cholinergic marketed for the treatment of airway hyperreactive diseases. While this compound is a potent anti-muscarinic agent, it is short acting, and thus must be administered as many as four times daily in order to provide relief for the COPD patient. In Europe and Asia, the long-acting anti-cholinergic Tiotropium Bromide (Spiriva©) was recently approved, however this product is currently not available in the United States. Thus, there remains a need for novel compounds that are capable of causing blockade at mAChRs which are long acting and can be administered once-daily for the treatment of airway hyperreactive diseases such as asthma and COPD.

Since mAChRs are widely distributed throughout the body, the ability to apply anti-cholinergics locally and/or topically to the respiratory tract is particularly advantageous, as it would allow for lower doses of the drug to be utilized. Furthermore, the ability to design topically active drugs that have long duration of action, and in particular, are retained either at the receptor or by the lung, would allow the avoidance of unwanted side effects that may be seen with systemic anti-cholinergic use.

### SUMMARY OF THE INVENTION

This invention provides for a method of treating a muscarinic acetylcholine receptor (mAChR) mediated disease, wherein acetylcholine binds to an M₃ mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent.

Compounds of Formula (I) useful in the present invention are represented by the structure:

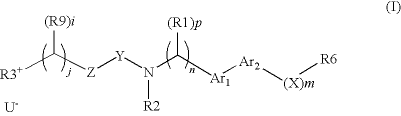

wherein

Ar1 and Ar2, are independently, selected from the group consisting of optionally substituted phenyl and optionally substituted monocyclic heteroaryl;

R6 is NR₇R₈, or an optionally substituted saturated or partially unsaturated 4-10 membered ring system in which one or more rings contain one or more secondary nitrogens, tertiary nitrogens, or quaternary ammonium nitrogens, and optionally contain one or more O, or S;

X is C(R1)p, or C(O); wherein, when X is C(R1)p m is an interger from 0 to 3; when X is C(O), m is 1;

p is an interger from 0 to 2;

i is an interger from 0 to 2;

n is an interger from 0 to 3;

j is an interger from 0 to 3;

Y is C(O), S(O)q, HNC(O), or OC(O); wherein, q is 1 or 2;

R1, R2, and R9 are independently selected from the group consisting of hydrogen, optionally substituted C₁-C₁₀ alkyl, optionally substituted C₃-C₁₀ cycloalkyl, optionally substituted C₃-C₁₀ cycloalkyl alkyl, optionally substituted heterocylic, optionally substituted heterocyclicalkyl, optionally substituted alkenyl, optionally substituted aryl, optionally substituted aryl alkyl, optionally substituted heteroaryl, and optionally substituted heteroaryl alkyl;

Z is selected from the group consisting of optionally substituted aryl, optionally substituted heteroaryl, optionally substituted alkenyl, optionally substituted C₁-C₁₀ alkyl, optionally substituted C₃-C₁₀ cycloalkyl, optionally substituted C₃-C₁₀ cycloalkyl alkyl, optionally substituted aryl alkyl, and optionally substituted heteroaryl alkyl;

R3⁺ is N⁺R₄R₅R₁₀, or an optionally substituted saturated or partially unsaturated 4-10 membered ring system in which one or more rings contain one or more quaternary ammonium nitrogens, and optionally contain one or more secondary or tertiary nitrogens, O, or S;

U⁻ is a pharmaceutically acceptable counter ion, selected from the group consisting of I⁻, Br⁻, Cl⁻, F⁻, CF3COO⁻, mesylate, and tosylate;

R₄, R₅, and R₁₀, are independently, selected from the group consisting of optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or any two or three of R₄, R₅, and R₁₀ together with the nitrogen to which they are attached form a 5 to 10 membered ring system which may optionally comprise an additional heteroatom selected from O, N and S;

R₇ and R₈, are independently, selected from the group consisting of hydrogen, optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or R₇ and R₈ together with the nitrogen to which they are attached form a 5 to 7 member ring which may optionally comprise an additional heteroatom selected from O, N and S;

or any other pharmaceutically acceptable salt thereof.

### DETAILED DESCRIPTION

The present invention includes all hydrates, solvates, complexes and prodrugs of the compounds of this invention. Prodrugs are any covalently bonded compounds that release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

For use herein the term “the aryl, heteroaryl, and heterocyclic containing moieties” refers to both the ring and the alkyl, or if included, the alkenyl rings, such as aryl, arylalkyl, and aryl alkenyl rings. The term “moieties” and “rings” may be interchangeably used throughout.

As used herein, “optionally substituted” unless specifically defined shall mean such groups as hydrogen; halogen, such as fluorine, chlorine, bromine or iodine; cyano; hydroxy; hydroxy substituted C₁₋₁₀ alkyl; cyano substituted C₁₋₁₀ alkyl; C₁₋₁₀ alkoxy, such as methoxy or ethoxy; S(O)_{m′} C₁₋₁₀ alkyl, wherein m′ is 0, 1 or 2, such as methyl thio, methyl sulfinyl or methyl sulfonyl; amino, mono & di-substituted amino, such as in the NR₇R₈ group; NHC(O)R₇; C(O)NR₇R₈; C(O)R₇; C(O)OH; S(O)₂NR₇R₈; NHS(O)₂R₇, C₁₀ alkyl, such as methyl, ethyl, propyl, isopropyl, or t-butyl; alkenyl, such as ethenyl, 1-propenyl, 2-propenyl, or 2-methyl-1-propenyl; halosubstituted C₁₋₁₀ alkyl, such CF₃; an optionally substituted aryl, such as phenyl, or an optionally substituted arylalkyl, such as benzyl or phenethyl, optionally substituted heterocylic, optionally substituted heterocyclic alkyl, optionally substituted heteroaryl, optionally substituted heteroaryl alkyl, wherein these aryl, heteroaryl, or heterocyclic moieties may be substituted one to two times by halogen; hydroxy; hydroxy substituted alkyl; C₁₋₁₀ alkoxy; S(O)_{m′}C₁₋₁₀ alkyl; amino, mono & di-substituted alkyl amino, such as in the NR₇R₈ group; C₁₋₁₀ alkyl, or halosubstituted C₁₋₁₀ alkyl, such as CF₃.

Suitable pharmaceutically acceptable salts are well known to those skilled in the art and include basic salts of inorganic and organic acids, such as hydrochloric acid, hydrobromic acid, sulphuric acid, phosphoric acid, methane sulphonic acid, ethane sulphonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid and mandelic acid.

The following terms, as used herein, refer to:

“halo” or “halogen”—chloro, fluoro, bromo and iodo.

“C₁₀alkyl” or “alkyl”—both straight and branched chain moieties of 1 to 10 carbon atoms, unless the chain length is otherwise limited, including, but not limited to, methyl, ethyl, n-propyl, isopropyl, n-butyl, sec butyl, isobutyl, tert-butyl, n-pentyl and the like.

“C₁-C₁₀ alkoxy” includes straight and branched chain radicals of the likes of —O—CH₃, —O—CH₂CH₃, and the n-propoxy, isopropoxy, n-butoxy, sec-butoxy, isobutoxy, tert-butoxy, pentoxy, and hexoxy, and the like.

“C₃-C₁₀ cycloalkyl” is used herein to mean cyclic moiety, including but not limited to cyclopropyl, cyclopentyl, cyclohexyl, and the like.

“alkenyl” is used herein at all occurrences to mean straight or branched chain moiety of 2-10 carbon atoms, unless the chain length is limited thereto, including, but not limited to ethenyl, 1-propenyl, 2-propenyl, 2-methyl-1-propenyl, 1-butenyl, 2-butenyl and the like.

“aryl”—phenyl and naphthyl;

“heteroaryl” (on its own or in any combination, such as “heteroaryloxy”, or “heteroaryl alkyl”)—a 5-10 membered aromatic ring system in which one or more rings contain one or more heteroatoms selected from the group consisting of N, O or S, such as, but not limited, to pyrrole, pyrazole, furan, thiophene, quinoline, isoquinoline, quinazolinyl, pyridine, pyrimidine, oxazole, tetrazole, thiazole, thiadiazole, triazole, imidazole, or benzimidazole.

“heterocyclic” (on its own or in any combination, such as “heterocyclicalkyl”)—a saturated or partially unsaturated 4-10 membered ring system in which one or more rings contain one or more heteroatoms selected from the group consisting of N, O, or S; such as, but not limited to, pyrrolidine, piperidine, piperazine, morpholine, tetrahydropyran, thiomorpholine, or imidazolidine. Furthermore, sulfur may be optionally oxidized to the sulfone or the sulfoxide.

“secondary nitrogen” is used herein to mean a nitrogen directly connected to one hydrogen, one optionally substituted carbon, and one optionally substituted carbon, C(O), or S(O)_{m′}; where in m′ is 1 or 2.

“tertiary nitrogen” is used herein to mean a nitrogen directly connected to two independent optionally substituted carbons, and one optionally substituted carbon, C(O), or S(O)_{m′}; where in m′ is 1 or 2.

“quaternary ammonium nitrogen” is used herein to mean a nitrogen directly connected to four independent optionally substituted carbons.

“arylalkyl” or “heteroarylalkyl” or “heterocyclicalkyl” is used herein to mean C₁₋₁₀ alkyl, as defined above, attached to an aryl, heteroaryl or heterocyclic moiety, as also defined herein, unless otherwise indicated.

“sulfinyl”—the oxide S(O) of the corresponding sulfide, the term “thio” refers to the sulfide, and the term “sulfonyl” refers to the fully oxidized S(O)₂ moiety.

The preferred compounds of Formula I include those compounds wherein:

Ar1 and Ar2, are independently, selected from the group consisting of optionally substituted phenyl and optionally substituted monocyclic heteroaryl;

R6 is an optionally substituted saturated or partially unsaturated 4-10 membered ring system in which one or more rings contain one or more secondary nitrogens, tertiary nitrogens, or quaternary ammonium nitrogens;

X is C(R1)p;

p is 2;

m is an interger from 0 to 3;

i is 2;

n is an interger from 1 to 3;

j is an interger from 0 to 3;

Y is C(O), or S(O)q; wherein, q is 1 or 2;

R1 is hydrogen

R9 is hydrogen

R2 is selected from the group consisting of hydrogen, optionally substituted C₁-C₁₀ alkyl, optionally substituted alkenyl, optionally substituted C₃-C₁₀ cycloalkyl, optionally substituted C₃-C₁₀ cycloalkyl alkyl, optionally substituted heterocylic, optionally substituted heterocyclicalkyl, optionally substituted aryl, optionally substituted aryl alkyl, optionally substituted heteroaryl, and optionally substituted heteroaryl alkyl;

Z is selected from the group consisting of optionally substituted aryl, optionally substituted heteroaryl, optionally substituted aryl alkyl, and optionally substituted heteroaryl alkyl;

R3⁺ is N⁺R₄R₅R₁₀, or an optionally substituted saturated or partially unsaturated 4-10 membered ring system in which one or more rings contain one or more quaternary ammonium nitrogens, and optionally contain one or more secondary or tertiary nitrogens, O, or S;

U⁻ is a pharmaceutically acceptable counter ion, selected from the group consisting of I⁻, Br⁻, Cl⁻, F⁻, CF3COO⁻, mesylate, and tosylate;

R₄, R₅, and R₁₀, are independently, selected from the group consisting of optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or any two or three of R₄, R₅, and R₁₀ together with the nitrogen to which they are attached form a 5 to 10 membered ring system which may optionally comprise an additional heteroatom selected from O, N and S;

or any other pharmaceutically acceptable salt thereof.

Even more preferred are those compounds where:

Ar1 and Ar2, are independently, selected from the group consisting of optionally substituted phenyl and optionally substituted monocyclic heteroaryl;

R6 is an optionally substituted saturated or partially unsaturated 5-8 membered ring system in which one or more rings contain one or more secondary or tertiary nitrogens;

X is C(R₁)p

p is 2;

m is 1;

i is 2;

n is 1;

j is 1, or 0;

Y is C(O), or S(O)q; wherein, q is 1 or 2;

R1 is hydrogen

R9 is hydrogen

R2 is selected from the group consisting of hydrogen, optionally substituted C₁-C₁₀ alkyl, optionally substituted alkenyl, optionally substituted C₃-C₁₀ cycloalkyl, optionally substituted C₃-C₁₀ cycloalkyl alkyl, optionally substituted heterocylic, optionally substituted heterocyclicalkyl, optionally substituted aryl alkyl, and optionally substituted heteroaryl alkyl;

Z is selected from the group consisting of optionally substituted phenyl, optionally substituted heteroaryl, optionally substituted phenyl alkyl, and optionally substituted heteroaryl alkyl;

R3⁺ is N⁺R₄R₅R₁₀, or an optionally substituted saturated or partially unsaturated 4-10 membered ring system in which one or more rings contain one or more quaternary ammonium nitrogens, and optionally contain one or more secondary or tertiary nitrogens, O, or S;

U⁻ is a pharmaceutically acceptable counter ion, selected from the group consisting of I⁻, Br⁻, Cl⁻, F⁻, CF3COO⁻, mesylate, and tosylate;

R₄, R₅, and R₁₀, are independently, selected from the group consisting of optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or any two or three of R₄, R₅, and R₁₀ together with the nitrogen to which they are attached form a 5 to 10 membered ring system which may optionally comprise an additional heteroatom selected from O, N and S;

or any other pharmaceutically acceptable salt thereof.

Illustrative compounds of Formula (I) include:
4-{[3-({[(6-fluoro-3′-{[(3S)-3-methyl-1-piperazinyl]methyl}-3-biphenylyl)methyl]amino}carbonyl)phenyl]methyl}-1,1-dimethylpiperazin-1-ium trifluoroacetate trifluoroacetic acid (1:3);
4-({3-[({[6-fluoro-3′-(1-piperazinylmethyl)-3-biphenylyl]methyl}amino)carbonyl]phenyl}methyl)-1,1-dimethylpiperazin-1-ium trifluoroacetate trifluoroacetic acid (1:3);
4-[(3-{[({3′-[(1S,4S)-2,5-diazabicyclo[2.2.1]hept-2-ylmethyl]-6-fluoro-3-biphenylyl}methyl)amino]carbonyl}phenyl)methyl]-1,1-dimethylpiperazin-1-ium trifluoroacetate trifluoroacetic acid (1:3);
1,1-dimethyl-4-({3-[({[6-(methyloxy)-3′-(1-piperazinylmethyl)-3-biphenylyl]methyl}amino)carbonyl]phenyl}methyl)piperazin-1-ium trifluoroacetate trifluoroacetic acid (1:3);
1,1-dimethyl-4-{[3-({[(6-(methyloxy)-3′-{[(3S)-3-methyl-1-piperazinyl]methyl}-3-biphenylyl)methyl]amino}carbonyl)phenyl]methyl}piperazin-1-ium trifluoroacetate trifluoroacetic acid (1:3);
4-({3-[({[3′-[(1S,4S)-2,5-diazabicyclo[2.2.1]hept-2-ylmethyl]-6-(methyloxy)-3-biphenylyl]methyl}amino)carbonyl]phenyl)methyl}-1,1-dimethylpiperazin-1-ium trifluoroacetate trifluoroacetic acid (1:3);
(1S,4S)-2-{[3-({[(6-fluoro-3′-{[(3S)-3-methyl-1-piperazinyl]methyl}-3-biphenylyl)methyl]amino}carbonyl)phenyl]methyl}-2-methyl-5-aza-2-azoniabicyclo[2.2.1]heptane trifluoroacetate trifluoroacetic acid (1:3);
1-{[3-({[(6-fluoro-3′-{[(3S)-3-methyl-1-piperazinyl]methyl}-3-biphenylyl)methyl]amino}carbonyl)phenyl]methyl}-1-methylpiperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1,1-dimethylhexahydro-1H-1,4-diazepin-1-ium trifluoroacetate trifluoroacetic acid (1:3);
1-{[3-({[(6-fluoro-3′-{[(3S)-3-methyl-1-piperazinyl]methyl}-3-biphenylyl)methyl]amino}carbonyl)phenyl]methyl}-1-methylhexahydro-1H-1,4-diazepin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-(3-hydroxypropyl)-1-methylpiperazin-1-ium trifluoroacetate trifluoroacetic acid (1:3);
(2R,5S)-4-{[3-({[(6-fluoro-3′-{[(3S)-3-methyl-1-piperazinyl]methyl}-3-biphenylyl)methyl]amino}carbonyl)phenyl]methyl}-1,1,2,5-tetramethylpiperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-[3-(methyloxy)propyl]piperazin-1-ium trifluoroacetate trifluoroacetic acid (1:3);
3-{[3-({[(6-fluoro-3′-{[(3S)-3-methyl-1-piperazinyl]methyl}-3-biphenylyl)methyl]amino}carbonyl)phenyl]methyl}-3-aza-6-azoniaspiro[5.6]dodecane trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-[(2E)-3-phenyl-2-propen-1-yl]piperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-propylpiperazin-1-ium trifluoroacetate trifluoroacetic acid (1:3);
2-(aminocarbonyl)-4-{[3-({[(6-fluoro-3′-{[(3S)-3-methyl-1-piperazinyl]methyl}-3-biphenylyl)methyl]amino}carbonyl)phenyl]methyl}-1,1-dimethylpiperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-(phenylmethyl)piperazin-1-ium trifluoroacetate trifluoroacetic acid (1:3);
3-{[3-({[(6-fluoro-3′-{[(3S)-3-methyl-1-piperazinyl]methyl}-3-biphenylyl)methyl]amino}carbonyl)phenyl]methyl}-3-aza-6-azoniaspiro[5.5]undecane trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-[2-(phenyloxy)ethyl]piperazin-1-ium trifluoroacetate trifluoroacetic acid (1:3);
1-ethyl-4-{[3-({[(6-fluoro-3′-{[(3S)-3-methyl-1-piperazinyl]methyl}-3-biphenylyl)methyl]amino}carbonyl)phenyl]methyl}-1-methylpiperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-(2-propen-1-yl)piperazin-1-ium trifluoroacetate trifluoroacetic acid (1:3);
(1S,4S)-5-{[3-({[(6-fluoro-3′-{[(3S)-3-methyl-1-piperazinyl]methyl}-3-biphenylyl)methyl]amino}carbonyl)phenyl]methyl}-2,2-dimethyl-5-aza-2-azoniabicyclo[2.2.1]heptane trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1,1,2-trimethylpiperazin-1-ium trifluoroacetate trifluoroacetic acid (1:3);
1-(3-cyanopropyl)-4-{[3-({[(6-fluoro-3′-{[(3S)-3-methyl-1-piperazinyl]methyl}-3-biphenylyl)methyl]amino}carbonyl)phenyl]methyl}-1-methylpiperazin-1-ium trifluoroacetate trifluoroacetic acid (1:3);
1-{[3-{([(6-fluoro-3′-{[(3S)-3-methyl-1-piperazinyl]methyl}-3-biphenylyl)methyl]amino}carbonyl)phenyl]methyl}-1,4-dimethylpiperidinium trifluoroacetate trifluoroacetic acid (1:2);
4-[2-(4-{[3-({[(6-fluoro-3′-{[(3S)-3-methyl-1-piperazinyl]methyl}-3-biphenylyl)methyl]amino}carbonyl)phenyl]methyl}-1-piperazinyl)ethyl]-4-methylmorpholin-4-ium trifluoroacetate trifluoroacetic acid (1:4);
1-{[3-({[(6-fluoro-3′-{[(3S)-3-methyl-1-piperazinyl]methyl}-3-biphenylyl)methyl]amino}carbonyl)phenyl]methyl}-4-formyl-1-methylpiperazin-1-ium trifluoroacetate trifluoroacetic acid (1:3);
1-{[3-({[(6-fluoro-3′-{[(3S)-3-methyl-1-piperazinyl]methyl}-3-biphenylyl)methyl]amino}carbonyl)phenyl]methyl}-1-methylpiperidinium trifluoroacetate trifluoroacetic acid (1:2);
4-{[3-({[(6-fluoro-3′-{[(3S)-3-methyl-1-piperazinyl]methyl}-3-biphenylyl)methyl]amino}carbonyl)phenyl]methyl}-4-methylmorpholin-4-ium trifluoroacetate trifluoroacetic acid (1:2);
[3-({[(6-fluoro-3′-{[(3S)-3-methyl-1-piperazinyl]methyl}-3-biphenylyl)methyl]amino}carbonyl)phenyl]-N,N,N-trimethylmethanaminium trifluoroacetate trifluoroacetic acid (1:2);
N-ethyl-N-{[3-({[(6-fluoro-3′-{[(3S)-3-methyl-1-piperazinyl]methyl}-3-biphenylyl)methyl]amino}carbonyl)phenyl]methyl}-N-methylethanaminium trifluoroacetate trifluoroacetic acid (1:2);
1-[2-(4-{[3-({[(6-fluoro-3′-{[(3S)-3-methyl-1-piperazinyl]methyl}-3-biphenylyl)methyl]amino}carbonyl)phenyl]methyl}-1-piperazinyl)ethyl]-1-methylpyrrolidinium iodide trifluoroacetic acid (1:3);
3-[{[3-({[(6-fluoro-3′-{[(3S)-3-methyl-1-piperazinyl]methyl}-3-biphenylyl)methyl]amino}carbonyl)phenyl]methyl}(methyl)amino]-1,1-dimethylpyrrolidinium iodide trifluoroacetic acid (1:3);
2-[{[3-({[(6-fluoro-3′-{[(3S)-3-methyl-1-piperazinyl]methyl}-3-biphenylyl)methyl]amino}carbonyl)phenyl]methyl}(methyl)amino]-N,N,N-trimethylethanaminium iodide trifluoroacetic acid (1:3);
4-(4-{[3-({[(6-fluoro-3′-{[(3S)-3-methyl-1-piperazinyl]methyl}-3-biphenylyl)methyl]amino}carbonyl)phenyl]methyl}-1-piperazinyl)-1,1-dimethylpiperidinium iodide trifluoroacetic acid (1:4);
2-(1-{[3-({[(6-fluoro-3′-{[(3S)-3-methyl-1-piperazinyl]methyl}-3-biphenylyl)methyl]amino}carbonyl)phenyl]methyl}-4-piperidinyl)-N,N,N-trimethylethanaminium iodide trifluoroacetic acid (1:3);
7-{[3-({[(6-fluoro-3′-{[(3S)-3-methyl-1-piperazinyl]methyl}-3-biphenylyl)methyl]amino}carbonyl)phenyl]methyl}-1,1-dimethyl-7-aza-1-azoniaspiro[4.4]nonane iodide trifluoroacetic acid (1:3);
(1-{[3-({[(6-fluoro-3′-{[(3S)-3-methyl-1-piperazinyl]methyl}-3-biphenylyl)methyl]amino}carbonyl)phenyl]methyl}-2-piperidinyl)-N,N,N-trimethylmethanaminium iodide trifluoroacetic acid (1:3);
3-[{[3-({[(6-fluoro-3′-{[(3S)-3-methyl-1-piperazinyl]methyl}-3-biphenylyl)methyl]amino}carbonyl)phenyl]methyl}(methyl)amino]-N,N,N-trimethyl-1-propanaminium iodide trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1,1-dimethylpiperidinium trifluoroacetate trifluoroacetic acid (1:1);
(2S)-4-[(5′-{[({3-[(1,1-dimethyl-4-piperidiniumyl)methyl]phenyl}carbonyl)amino]methyl}-2′-fluoro-3-biphenylyl)methyl]-1,1,2-trimethylpiperazin-1-ium dibromide; and
4-{[3-({[(3′-{[(3S)-3,4-dimethyl-1-piperazinyl]methyl}-6-fluoro-3-biphenylyl)methyl]amino}carbonyl)phenyl]methyl}-1,1-dimethylpiperidinium trifluoroacetate trifluoroacetic acid (1:1);

or any other pharmaceutically acceptable salt thereof.

### Methods of Preparation

### Preparation

The compounds of Formula (I) may be obtained by applying synthetic procedures, some of which are illustrated in the Schemes below. The synthesis provided for these Schemes is applicable for producing compounds of Formula (I) having a variety of different R₁, R₂, R₃, R₄, X, Y, and Z, which are reacted, employing substituents which are suitable protected, to achieve compatibility with the reactions outlined herein. Subsequent deprotection, in those cases, then affords compounds of the nature generally disclosed. While some Schemes are shown with specific compounds, this is merely for illustration purpose only.

### Preparation 1

As shown in Scheme 1, bromo benzylamines 1 were loaded onto 2,6-dimethoxy-4-polystyrenebenzyloxy-benzaldehyde (DMHB resin) via reductive amination. The resin-bound amines 2 were reacted with 3-formylbenzoic acid to yield amides 3, which were reductively aminated with various amines NHR1R2, to yield amines 4. Suzuki coupling of 4 with (3-formylphenyl)boronic acid gave biphenylaldehydes 5. Quaternization of resin bound amines 5 with methyl iodide, other alkyl halides or various alkyl dihalides (for example, α, ω dihalides), gave resin bound quaternary ammonium salts 6, which were then subject to reductive amination with amines NHR3R4, followed by cleavage, affording desired products 7.

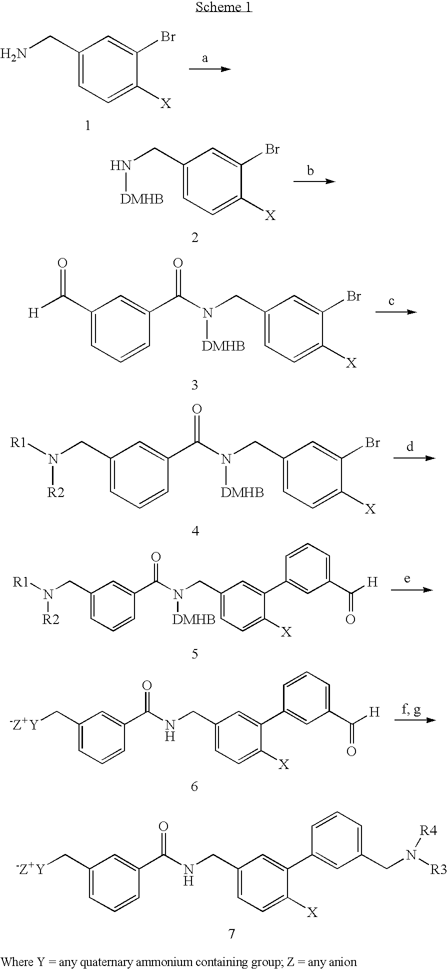

Conditions: a) DMHB resin, Na(OAc)₃BH, acetic acid, 1-methyl-2-pyrrolidinone (NMP), rt; b) 3-formylbenzoic acid, 1,3-diisopropylcarbodiimide (DIC), 1,2-dichloroethane (DCE): dimethylformamide (DMF)=1:1, rt; c) NHR1R2, Na(OAc)₃BH, Na₂SO₄, dichloroethane (DCE), rt; d) (3-formylphenyl)boronic acid, Pd(PPh₃)₄, Cs₂CO₃, dimethoxyethane (DME), 80° C.; e) MeI, various alkyl halides, or various alkyl dihalides, acetonitrile, temperatures between room temperature and 90° C. depending on substrate; f) NHR3R4, Na(OAc)₃BH, Na₂SO₄, DCE, rt; g) 50% of trifluoroacetic acid (TFA) in DCE, rt.

### SYNTHETIC EXAMPLES

The invention will now be described by reference to the following Examples which are merely illustrative and are not to be construed as a limitation of the scope of the present invention. Most reagents and intermediates are commercially available or are prepared according to procedures in the literature. The preparation of intermediates not described in the literature is illustrated below.

Flash column chromatography was carried out using Merck 9385 silica unless stated otherwise.

LC/MS analyses were conducted under the following conditions:
Column: 3.3 cm×4.6 mm ID, 3 um ABZ+PLUS
Flow Rate: 3 ml/min
Injection Volume: 5 μl
Temp: Room temperature
Solvents: A: 0.1% Formic Acid+10 mMolar Ammonium Acetate.
   B: 95% Acetonitrile+0.05% Formic Acid

Gradient:

| Time | A % | B % |
|---|---|---|
| 0.00 | 100 | 0 |
| 0.70 | 100 | 0 |
| 4.20 | 0 | 100 |
| 5.30 | 0 | 100 |
| 5.50 | 100 | 0 |

The Mass Directed Automated Preparative (MDAP) was conducted under the conditions described in System A or in System B:

### System A: Formate Salts

The preparative column used was a Supelcosil ABZplus (10 cm×2.12 cm internal diameter; particle size 5 m)
UV detection wavelength: 200-320 nM
Flow rate: 20 ml/min
Injection Volume: 0.5 ml
Solvent A: 0.1% formic acid
Solvent B: 95% acetonitrile+0.05% formic acid

### System B TFA Salts

The preparative column used was a Supelcosil ABZplus (10 cm×2.12 cm internal diameter; particle size 5 m)
UV detection wavelength: 200-320 nM
Flow rate: 20 ml/min
Injection Volume: 0.5 ml
Solvent A: water+0.1% trifluoroacetic acid
Solvent B: acetonitrile+0.1% trifluoroacetic acid

The Gilson preparatory HPLC was conducted under the following conditions:
Column: 75×33 mm I.D., S-5 um, 12 nm
Flow rate: 30 mL/min
Injection Volume: 0.800 mL
Room temperature
Solvent A: 0.1% trifluoroacetic acid in water
Solvent B: 0.1% trifluoroacetic acid in acetonitrile

### Example 1

### Preparation of 4-{[3-({[(6-fluoro-3′-{[(3S)-3-methyl-1-piperazinyl]methyl}-3-biphenylyl)methyl]amino}carbonyl)phenyl]methyl}-1,1-dimethylpiperazin-1-ium trifluoroacetate trifluoroacetic acid (1:3)

### a) DMHB Resin Bound 3-bromo-4-fluoro-benzylamine

To a mixture of DMHB resin (10 g, 1.5 mmol/g loading, 15 mmol) in N-methylpyrrolidine (NMP, 150 mL), was added 3-bromo-4-fluoro-benzylamine (15.5 g, 75 mmol), acetic acid (15 mL, 1% v/v), and sodium triacetoxyborohydride (19 g, 90 mmol). The mixture was shaken at rt for overnight and was then washed with NMP (200 mL×2), dichloromethane (DCM) (200 mL×2), MeOH (200 mL×2) and DCM (200 mL×2). The resulting resin was dried in vacuum oven at 20° C. for overnight to yield DMHB resin bound 3-bromo-4-fluoro-benzylamine (15 mmol).

### b) DMHB resin-bound N-[(3-bromo-4-fluorophenyl)methyl]-3-formylbenzamide

To a mixture of DMHB resin-bound 3-bromo-4-fluoro-benzylamine (200 mg, 1.15 mmol/g (theoretical loading), 0.23 mmol) in DCE/DMF (1:1, 8 mL) was added 3-formylbenzoic acid (350 mg, 2.3 mmol) and DIC (0.36 mL, 2.3 mmol). The mixture was shaken at rt for overnight and was then washed with DMF (20 mL×2), DCM (20 mL×2), MeOH (20 mL×2) and DCM (20 mL×2). The resulting resin was dried in vacuum oven at 20° C. for overnight to yield DMHB resin-bound N-[(3-bromo-4-fluorophenyl)methyl]-3-formylbenzamide (0.23 mmol). An analytical amount of the resin was cleaved with 50% of TFA in DCE for 10 min. The resulting solution was concentrated in vacuo and dissolved in 0.5 mL of CH₃CN. MS (ESI): 336 [M+H]⁺.

### c) DMHB resin-bound N-[(3-bromo-4-fluorophenyl)methyl]-3-[(4-methyl-1-piperazinyl)methyl]benzamide

To a mixture of DMHB resin-bound bound N-[(3-bromo-4-fluorophenyl)methyl]-3-formylbenzamide (200 mg, 0.99 mmol/g, 0.198 mmol) in 10 mL of DCE was added Na₂SO₄ (0.141 g, 0.99 mmol) and 1-methylpiperazine (0.1 g, 0.99 mmol). After shaking for 10 min, Na(OAc)₃BH (0.252 g, 1.19 mmol) was added. After being shaken at rt for overnight, the resin was washed with tetrahydrofuran (THF) (20 mL×2), THF:H₂O (1:1, 20 mL×2), H₂O (20 mL×2), THF:H₂O (1:1, 20 mL×2), THF (20 mL×2), DCM (20 mL×2) and dried in vacuum oven at 20° C. for overnight to afford DMHB resin-bound N-[(3-bromo-4-fluorophenyl)methyl]-3-[(4-methyl-1-piperazinyl)methyl]benzamide (0.198 mmol). An analytical amount of the resin was cleaved with 50% of TFA in DCE for 10 min. The resulting solution was concentrated in vacuo and dissolved in 0.5 mL of CH₃CN. MS (ESI): 420 [M+H]⁺.

### d) DMHB resin-bound N-[(6-fluoro-3′-formyl-3-biphenylyl)methyl]-3-[(4-methyl-1-piperazinyl)methyl]benzamide

To a mixture of DMHB resin-bound N-[(3-bromo-4-fluorophenyl)methyl]-3-[(4-methyl-1-piperazinyl)methyl]benzamide (200 mg, 0.92 mmol/g, 0.184 mmol) in 5 mL DME was added 3-formylphenyl boronic acid (83 mg, 0.55 mmol), 2 M Cs₂CO₃ aqueous solution (0.275 mL, 0.55 mmol), and Pd(PPh₃)₄ (43 mg, 0.0368 mmol). After being purged with argon for 5-10 min, the mixture was heated at 80° C. under argon for 16 h. The resulting resin was washed with THF (20 mL×2), THF:H₂O (1:1, 20 mL×2), H₂O (20 mL×2), THF:H₂O (1:1, 20 mL×2), THF (20 mL×2), DCM (20 mL×2), and dried in vacuum oven at 20° C. for overnight to afford DMHB resin-bound N-[(6-fluoro-3′-formyl-3-biphenylyl)methyl]-3-[(4-methyl-1-piperazinyl)methyl]benzamide (0.184 mmol). An analytical amount of the resin was cleaved with 50% of TFA in DCM for 10 min. The resulting solution was concentrated in vacuo and dissolved in 0.5 mL of CH₃CN. MS (ESI): 446 [M+H]⁺.

### e) DMHB resin-bound 4-{[3-({[(6-fluoro-3′-formyl-3-biphenylyl)methyl]amino}carbonyl)phenyl]methyl}-1,1-dimethylpiperazin-1-ium iodide

To a mixture of DMHB resin-bound N-[(6-fluoro-3′-formyl-3-biphenylyl)methyl]-3-[(4-methyl-1-piperazinyl)methyl]benzamide (200 mg, 0.91 mmol/g, 0.182 mmol) in 7 mL acetonitrile was added methyl iodide (390 mg, 2.73 mmol), and the resin was shaken for overnight. The resulting resin was washed with CH₃CN (20 mL×2), DCM (20 mL×2), MeOH (20 mL×2), DCM (20 mL×2), MeOH (20 mL×2), DCM (20 mL×2), and dried in vacuum oven at 20° C. for overnight to afford DMHB resin-bound 4-{[3-({[(6-fluoro-3′-formyl-3-biphenylyl)methyl]amino}carbonyl)phenyl]methyl}-1,1-dimethylpiperazin-1-ium iodide (0.182 mmol). An analytical amount of the resin was cleaved with 50% of TFA in DCM for 10 min. The resulting solution was concentrated in vacuo and dissolved in 0.5 mL of CH₃CN. MS (ESI): 460 [M]⁺.

### f, g) 4-{[3-({[(6-fluoro-3′-{[(3S)-3-methyl-1-piperazinyl]methyl}-3-biphenylyl)methyl]amino}carbonyl)phenyl]methyl}-1,1-dimethylpiperazin-1-ium trifluoroacetate trifluoroacetic acid (1:3)

To a mixture of DMHB resin-bound 4-{[3-({[(6-fluoro-3′-formyl-3-biphenylyl)methyl]amino}carbonyl)phenyl]methyl}-1,1-dimethylpiperazin-1-ium iodide (200 mg, 0.81 mmol/g, 0.162 mmol) in 17 mL of DCE was added Na₂SO₄ (0.13 g, 0.9 mmol) and (S)-2-methylpiperazine (0.092 g, 0.9 mmol). After shaking for 10 min, Na(OAc)₃BH (0.229 g, 1.08 mmol) was added. After being shaken at rt for overnight, the resin was washed with THF (20 mL×2), THF:H₂O (1:1, 20 mL×2), H₂O (20 mL×2), THF:H₂O (1:1, 20 mL×2), THF (20 mL×2), DCM (20 mL×2) and dried in vacuum oven at 20° C. for overnight. The resulting resin was cleaved with 5 mL of 50% of TFA in DCE for 30 min and treated again with 5 mL of 50% of TFA in DCE for 30 min. The combined cleavage solution was concentrated in vacuo. The residue was dissolved in dimethylsulfoxide (DMSO) and purified using a Gilson semi-preparative HPLC system with a YMC ODS-A (C-18) column 50 mm by 30 mm ID, eluting with 10% B to 90% B in 3.2 min, hold for 1 min where A=H₂O (0.1% trifluoroacetic acid) and B═CH₃CN (0.1% trifluoroacetic acid) pumped at 25 mL/min, to produce 4-{[3-({[(6-fluoro-3′-{[(3S)-3-methyl-1-piperazinyl]methyl}-3-biphenylyl)methyl]amino}carbonyl)phenyl]methyl}-1,1-dimethylpiperazin-1-ium trifluoroacetate trifluoroacetic acid (1:3) (white powder, 113 mg, 38% over 6 steps). MS (ESI): 544 [M]⁺.

Proceeding in a similar manner, but replacing 1-methylpiperazine with appropriate amines, and/or replacing (S)-2-methylpiperazine with appropriate amines, and/or replacing 3-bromo-4-fluoro-benzylamine with appropriate bromobenzylamines, and/or replacing methyl iodide with appropriate alkyl halides or alkyl dihalides, the compounds listed in Tables 1 and 2 were prepared.

In the case of examples 7, 8, and 10, the amine used in the step c) was a mono t-butoxy carbonyl (BOC) protected amine. The BOC group was later removed during the step g).

In the case of examples 2, 3, 4, and 6, the amine used in the step f) was a BOC-protected amine. The BOC group was later removed during the step g).

TABLE 1

| Example | Compound | MS [M]⁺ |
|---|---|---|
| 2 | 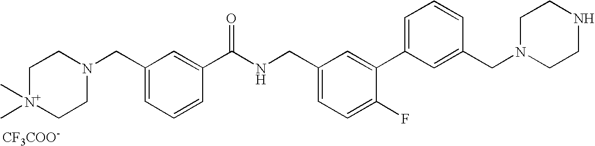 | 530 |
| 3 | 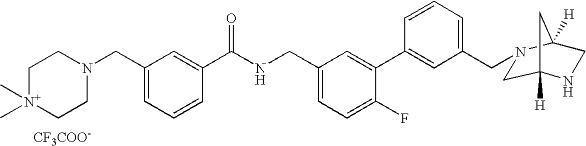 | 542 |
| 4 | 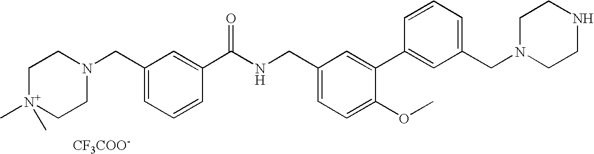 | 542 |
| 5 | 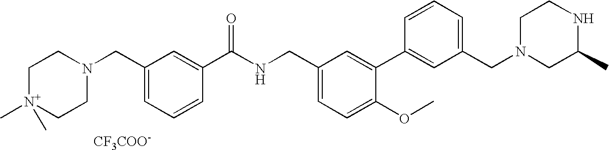 | 556 |
| 6 | 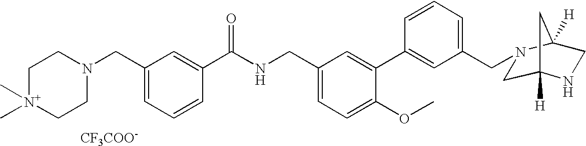 | 554 |
| 7 | 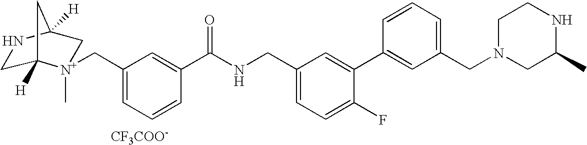 | 542 |
| 8 | 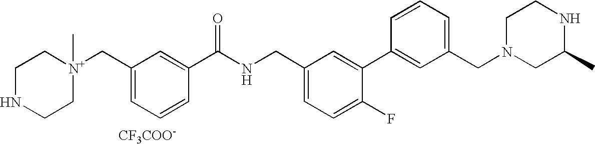 | 530 |
| 9 | 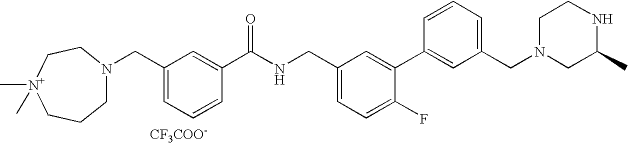 | 558 |
| 10 | 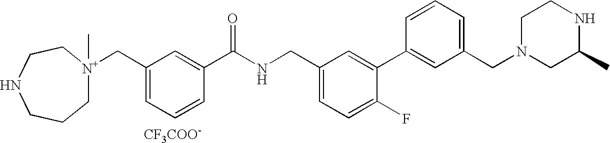 | 544 |
| 11 | 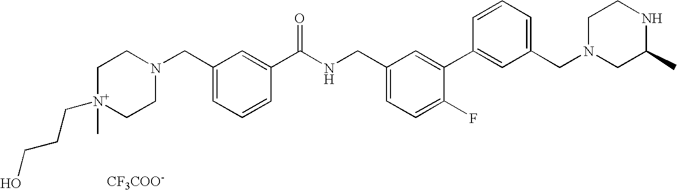 | 588 |
| 12 | 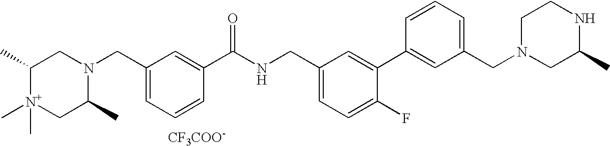 | 572 |
| 13 | 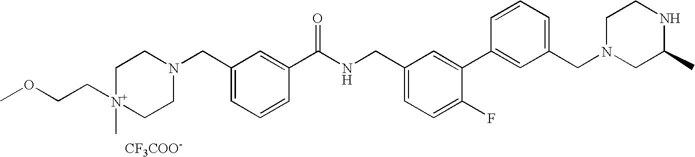 | 602 |
| 14 | 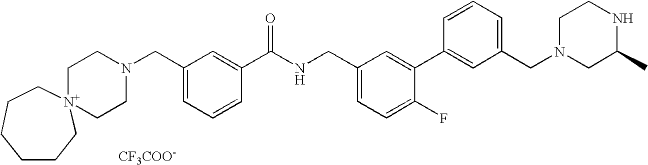 | 598 |
| 15 | 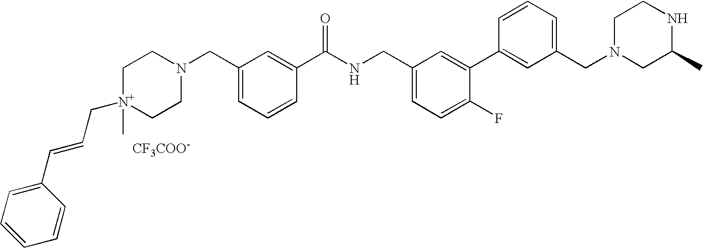 | 646 |
| 16 | 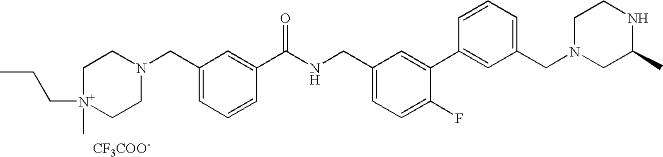 | 572 |
| 17 | 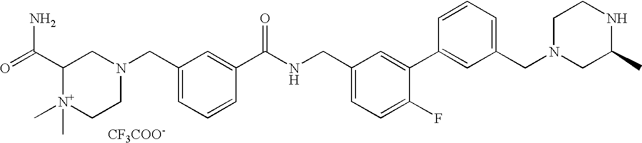 | 587 |
| 18 | 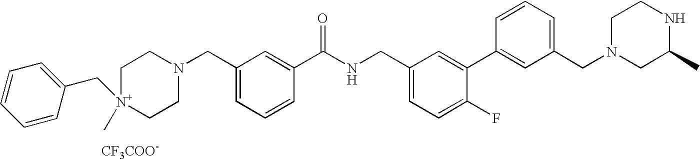 | 620 |
| 19 | 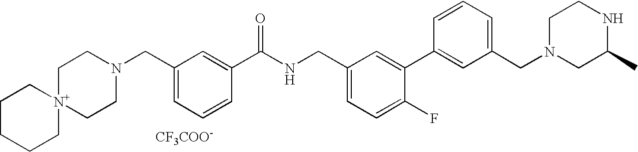 | 584 |
| 20 | 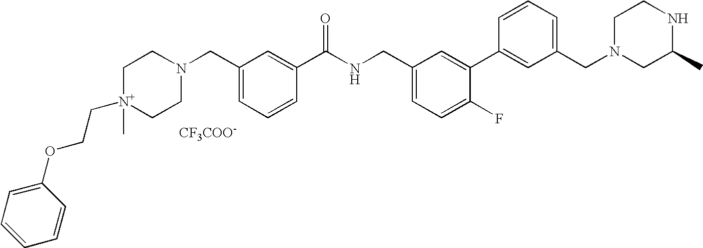 | 650 |
| 21 | 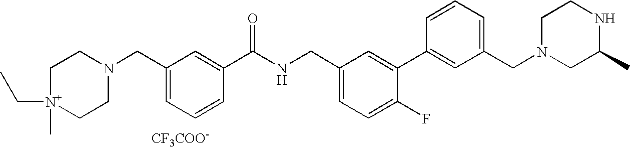 | 558 |
| 22 | 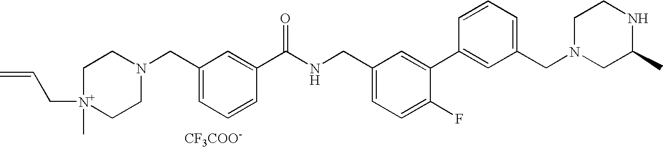 | 570 |
| 23 | 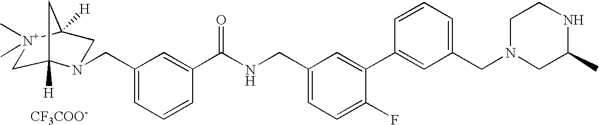 | 556 |
| 24 | 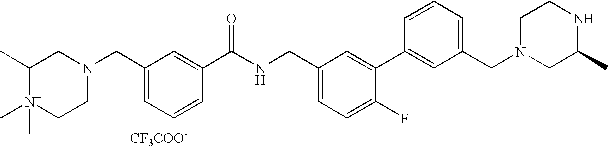 | 558 |
| 25 | 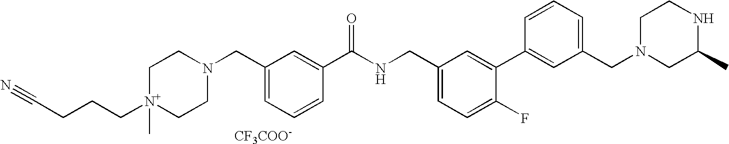 | 597 |
| 26 | 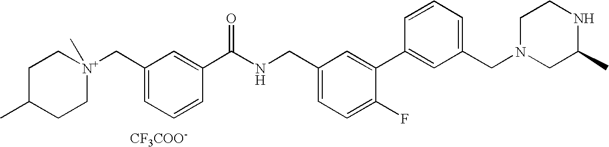 | 543 |
| 27 | 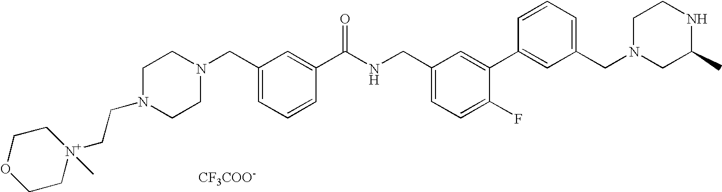 | 643 |
| 28 | 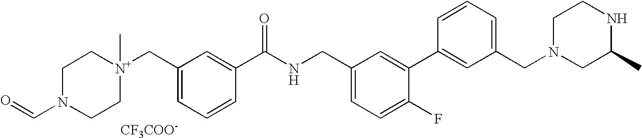 | 558 |
| 29 | 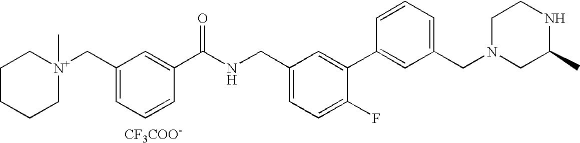 | 529 |
| 30 | 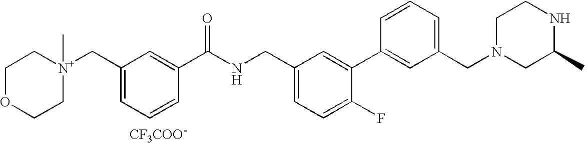 | 531 |
| 31 | 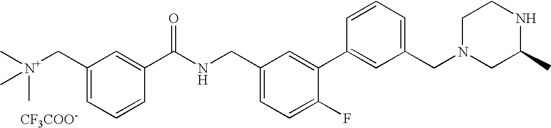 | 489 |
| 32 | 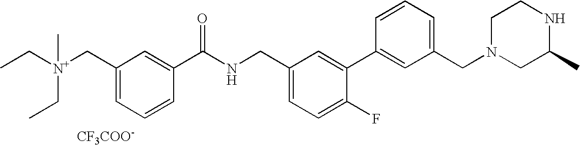 | 517 |

TABLE 2

| Example | Compound | MS [M + I + H]⁺ |
|---|---|---|
| 33 | 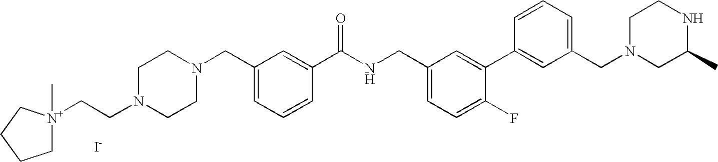 | 755 |
| 34 | 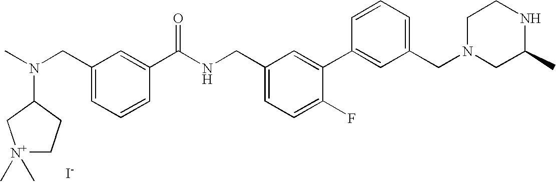 | 686 |
| 35 | 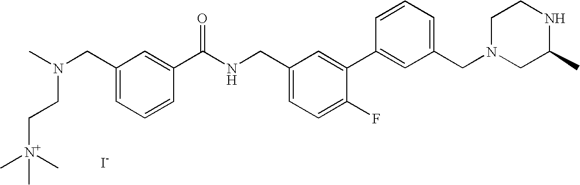 | 674 |
| 36 | 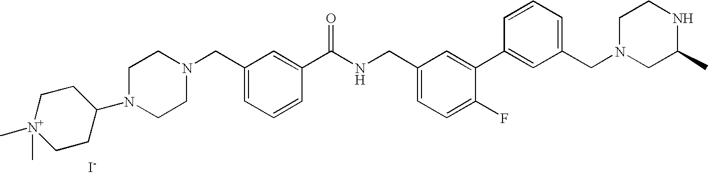 | 755 |
| 37 | 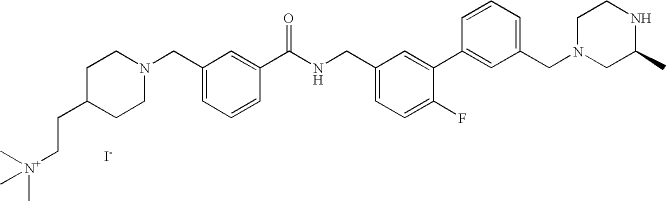 | 728 |
| 38 | 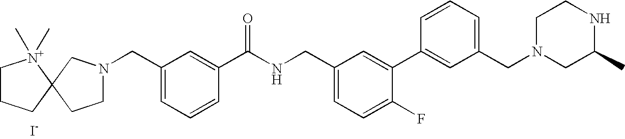 | 712 |
| 39 | 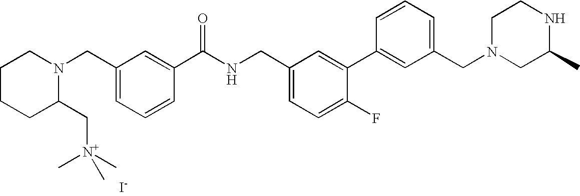 | 714 |
| 40 | 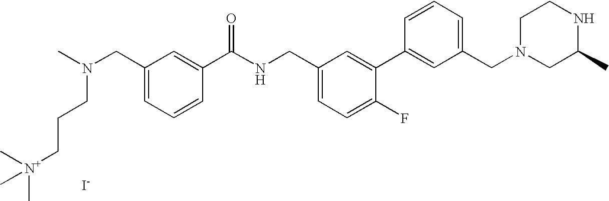 | 688 |

### Preparation 2

The compounds of structure 15 and 16 were prepared in solution phase following the route outlined in Scheme 2. Firstly, reductive amination of the benzaldehyde 8 with the N-protected piperazine 9 gave the tertiary amine 11. Coupling of 11 with the boronic acid 10 using the Suzuki reaction gave the biphenyl derivative 12. Further reduction of the nitrile moiety with borane yielded the primary amine 13. Subsequent coupling of 13 to the commercially available benzoic acid 17 gave the corresponding amide 1.4. Deprotection of the Boc group on the piperidine nitrogen of 14 followed by reductive amination, reaction with methyl iodide and removal of the benzyloxycarbonyl protecting group led to the quaternary salt 15. Further reductive amination of the terminal nitrogen of the piperazine group led to compound 16.

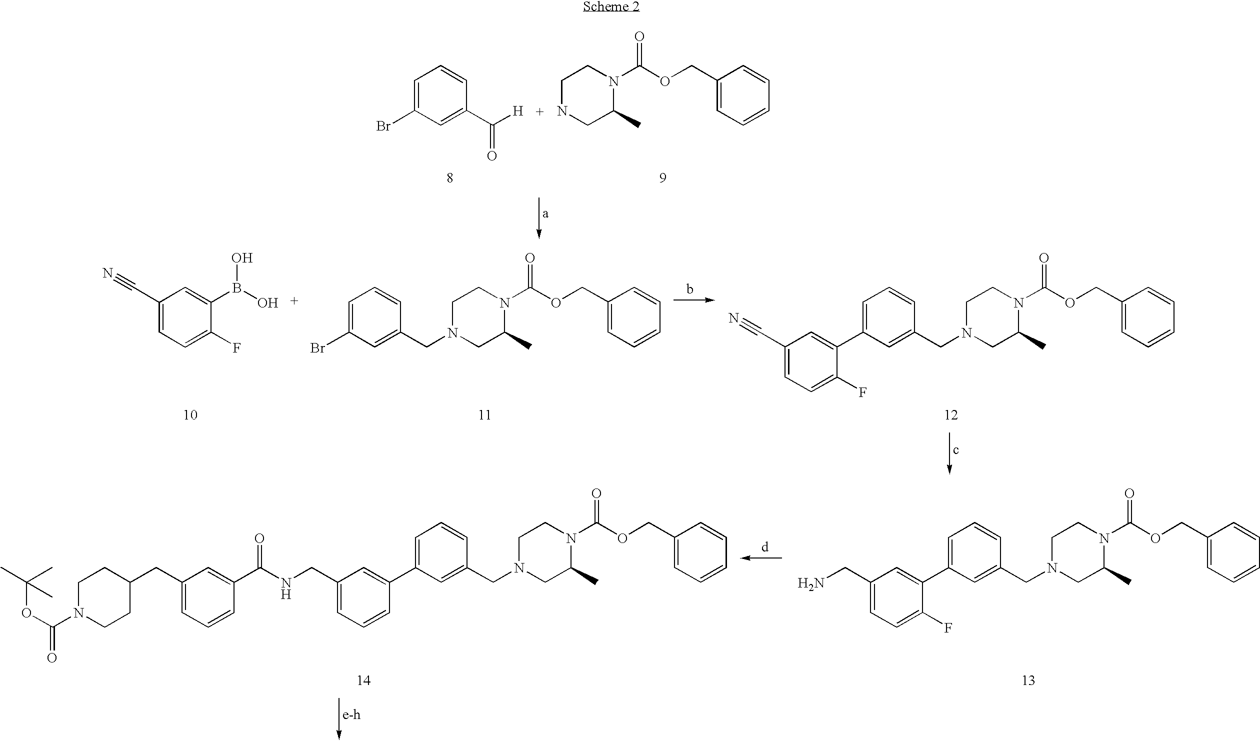

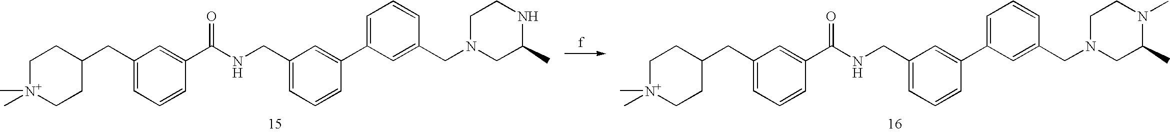

Conditions: a) NaB(OAc)₃H, DCM, rt; b) Pd(PPh₃)₄, Na₂CO₃, DME, 78° C.; c) BH₃, THF; d) HATU, 3-[(1-{[(1,1-dimethylethyl)oxy]carbonyl}-4-piperidinyl)methyl]benzoic acid 17, DIPEA, DMF; e) HCl, dioxane; f) HCHO, NaBH₄; g) MeI, acetone; h) 6N aq. HCl, MeOH.

### Intermediate 1

### phenylmethyl (2S)-4-[(3-bromophenyl)methyl]-2-methyl-1-piperazinecarboxylate

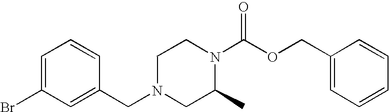

### Preparation of (3S)-1-[(3-Bromophenyl)methyl]-3-methylpiperazine

To a solution of (2S)-2-methyl-piperazine (1 g, 10 mmol) in CH₂Cl₂ (5 mL) was added 3-bromo benzaldehyde (1.85 g, 10 mmol) and NaB(OAc)₃H (3.18 g, 1.5 mmol). The resulting mixture was stirred for 12 hours, diluted with dichloromethane (25 mL) and washed with brine (5 mL). The organic layer was collected, dried over Na₂SO₄ and concentrated under vacuum. Purification by Gilson reverse phase HPLC, eluting with acetonitrile/water/0.1% TFA (5 to 70%, v/v, over 12 min) of the crude material afforded the title compound (2.0 g, 40%): LC/MS: m/z, 269 (M+H), 1.28 min.

### Preparation of phenylmethyl (2S)-4-[(3-bromophenyl)methyl]-2-methyl-1-piperazinecarboxylate

To a solution of (3S)-1-[(3-Bromophenyl)methyl]-3-methylpiperazine (250 mg, 0.5 mmol), TEA (0.5 mL, 3.5 mmol), and DMAP (12 mg, 0.1 mmol) in 1 mL of dry DMSO was added dropwise benzyl chloroformate (0.34 mL, 2.25 mmol) at 10° C. while stirring. The mixture was then heated and stirred at 50° C. for 1.5 h. After cooling to room temperature, 15 ml of ethyl acetate and 5 ml of saturated NaHCO₃ were added. The organic layer was separated, concentrated under vacuum and purified by Gilson reverse phase HPLC, eluting with acetonitrile/water/0.1% TFA (10/90 to 70/30, v/v, over 12 min), to give the title compound (180 mg, 70%). LC/MS: m/z, 403 (M+H), 1.74 min.

### Intermediate 2

### Phenylmethyl (2S)-4-[(5′-cyano-2′-fluoro-3-biphenylyl)methyl]-2-methyl-1-piperazinecarboxylate

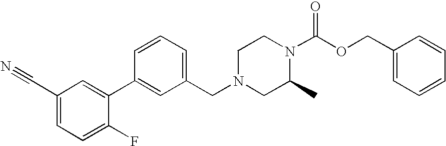

To the solution of (5-cyano-2-fluorophenyl)boronic acid (660 mg, 4 mmol) in dioxane/H₂O (40 mL/13.3 mL) were added phenylmethyl (2S)-4-[(3-bromophenyl)methyl]-2-methyl-1-piperazinecarboxylate (1.2 g, 4 mmol), K₂CO₃ (2.2 g mg, 16 mmol) and Pd(PPh₃)₄ (230 mg, 0.2 mmol). The resulting solution was irradiated in a microwave reactor at 150° C. for 20 minutes then diluted with EtOAc (5 mL). The organic layer was collected and the aqueous layer was extracted with EtOAc (2×5 mL). The organic layers were combined, dried over Na₂SO₄, filtered and concentrated. The residue was purified by Gilson HPLC, eluting with acetonitrile/water/0.1% TFA (10/90 to 90/10, v/v, over 12 min), to give the title compound (708 mg, 92%). LC/MS: m/z, 444 (M+H), 1.93 min.

### Intermediate 3

### Phenylmethyl (2S)-4-{[5′-(aminomethyl)-2′-fluoro-3-biphenylyl]methyl}-2-methyl-1-piperazinecarboxylate

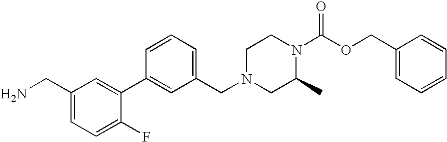

A solution of phenylmethyl (2S)-4-[(5′-cyano-2′-fluoro-3-biphenylyl)methyl]-2-methyl-1-piperazinecarboxylate (708 mg, 1.60 mmol) in THF (10 mL) was flushed with Ar. for 15 minutes. Borane (5.6 mL of a 1 M solution in THF, 5.6 mmol) was slowly added and the reaction mixture was allowed to stir at room temperature for 12 hours. The reaction was quenched slowly with 1 N HCl (1 mL) and allowed to stir for 2 hours at rt. After neutralization to pH >10 with 2N NaOH, the reaction mixture was extracted with EtOAc (2×15 mL). The combined organic layers were dried over MgSO₄, filtered, and concentrated under vacuum. The crude residue was placed onto a aminopropyl SPE silica cartridge (10 g) and eluted with the following sequence: 50% hexane/50% EtOAc (3×20 mL), 100% MeOH/90% DCM (3×20 mL). The methanol fractions were combined and concentrated to give the title compound (660 mg, 92%). LC/MS: m/z, 448 (M+H), 1.63 min.

### Intermediate 4

### Phenylmethyl (2S)-4-[(5′-{[({3-[(1-{[(1,1-dimethylethyl)oxy]carbonyl}-4-Piperidinyl)methyl]phenyl}carbonyl)amino]methyl}-2′-fluoro-3-biphenylyl)methyl]-2-methyl-1-piperazinecarboxylate

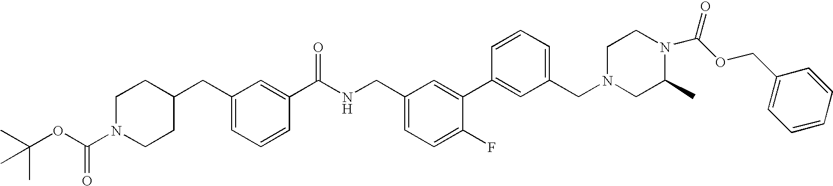

To a solution of 3-[(1-{[(1,1-dimethylethyl)oxy]carbonyl}-4-piperidinyl)methyl]benzoic acid (108 mg, 0.34 mmol) in dry DMF (2 mL) were added phenylmethyl (2S)-4-{[5′-(aminomethyl)-2′-fluoro-3-biphenylyl]methyl}-2-methyl-1-piperazinecarboxylate (150 mg, 0.334 mmol), DIPEA (0.1 mL, 0.7 mmol), HATU (142 mg, 0.37 mmol) and HOBt (150 mg, 1.1 mmol). The reaction mixture was stirred at room temperature for 2 h. followed by addition of saturated aq. Na₂CO₃ (1 mL) and EtOAc (5 mL). The organic layer was separated, dried over Na₂SO₄, and filtered. The filtrate was concentrated and the residue was purified on a 5 g aminopropyl SPE cartridge, eluting with DCM (3×5 mL), EtOAc (3×5 mL), and MeOH (3×5 mL). The product was recovered after evaporation of the DCM fractions (130 mg, 80%). LC/MS: m/z, 749 (M+H), 2.32 min.

### Intermediate 5

### Phenylmethyl (2S)-4-({2′-fluoro-5′-[({[3-(4-piperidinylmethyl)phenyl]carbonyl}amino)methyl]-3-biphenylyl}methyl)-2-methyl-1-piperazinecarboxylate

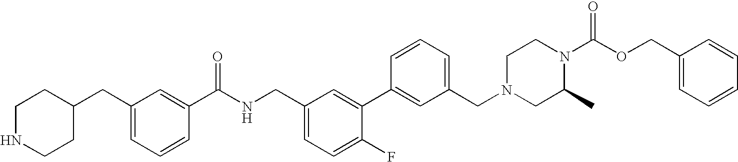

To a solution of phenylmethyl (2S)-4-[(5′-{[({3-[(1-{[(1,1-dimethylethyl)oxy]carbonyl}-4-piperidinyl)methyl]phenyl}carbonyl)amino]methyl}-2′-fluoro-3-biphenylyl)methyl]-2-methyl-1-piperazinecarboxylate (673 mg, 0.90 mmol) in 5 mL of 1,4-dioxane was added 5 mL of 4M HCl in 1,4-dioxane. The mixture was stirred at room temperature for 30 min. After removal of the solvent, the crude was purified by Gilson reverse phase HPLC, eluting with acetonitrile/water/0.1% TFA (10/90 to 70/30, v/v, over 12 min), to give the title compound (390 mg, 67%). LC/MS: m/z, 649 (M+H), 1.69 min.

### Intermediate 6: Phenylmethyl (2S)-4-[(2′-fluoro-5′-{[({3-[(1-methyl-4-piperidinyl)methyl]phenyl}carbonyl)amino]methyl}-3-biphenylyl)methyl]-2-methyl-1-piperazinecarboxylate

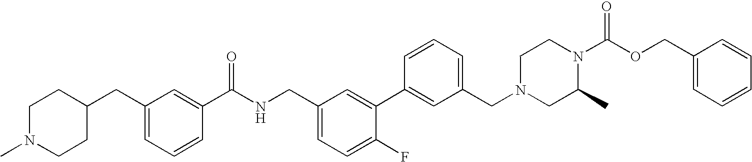

To a solution of phenylmethyl (2S)-4-({2′-fluoro-5′-[({[3-(4-piperidinyl)phenyl]carbonyl}amino)methyl]-3-biphenylyl}methyl)-2-methyl-1-piperazinecarboxylate in 5 mL of MeOH, was added dropwise formaldehyde (37%, 170 mg, 2.04 mmol). The mixture was stirred for 12 hours, the solvent was removed under vacuum, and the residue was purified on a 2 g aminopropyl SPE cartridge, eluting with DCM (3×5 mL), EtOAc (3×5 mL), and MeOH (3×5 mL). The EtOAc fractions were combined ad evaporated to give the title compound (240 mg, 71%). LC/MS: m/z, 663 (M+H), 2.02 min.

### Intermediate 7: 4-[(3-{[({6-fluoro-3′-[((3S)-3-methyl-4-{[(phenylmethyl)oxy]carbonyl}-1-piperazinyl)methyl]-3-biphenylyl}methyl)amino]carbonyl}phenyl)methyl]-1,1-dimethylpiperidinium iodide

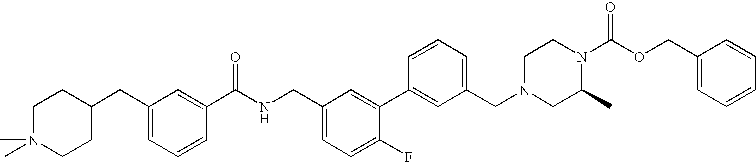

To a solution of phenylmethyl (2S)-4-[(2′-fluoro-5′-{[({3-[(1-methyl-4-piperidinyl)methyl]phenyl}carbonyl)amino]methyl}-3-biphenylyl)methyl]-2-methyl-1-piperazinecarboxylate (95 mg, 0.14 mmol) in acetone (2 mL) was added iodomethane (0.174 mL, 2.8 mmol). The resulting mixture was allowed to stir at room temperature for 16 hours then the solvent was evaporated under vacuum to give the title compound as a yellow solid (120 mg). LC/MS: m/z, 677 (M+H), 1.50 min.

### Example 41

### 4-{[3-({[(6-fluoro-3′-{[(3S)-3-methyl-1-piperazinyl]methyl}-3-biphenylyl)methyl]amino}carbonyl)phenyl]methyl}-1,1-dimethylpiperidinyl trifluoroacetate trifluoroacetic acid (1:1)

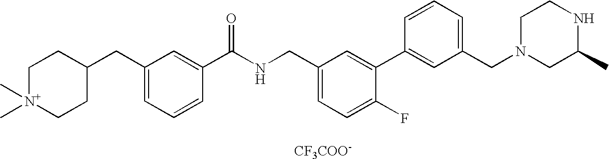

To a solution of 4-[(3-{[({6-Fluoro-3′-[((3S)-3-methyl-4-{[(phenylmethyl) oxy]carbonyl}-1-piperazinyl)methyl]-3-biphenylyl}methyl)amino carbonyl}phenyl)methyl]-1,1-dimethylpiperidinyl iodide (100 mg, 0.124 mmol) in MeOH (2 mL), was added 6N aq. HCl (2 mL). The mixture was allowed to react in a microwave oven at 150° C. for 3 min. After removal of the solvent under vacuum, the residue was purified by Gilson reverse phase HPLC, eluting with acetonitrile/water/0.1% TFA (10/90 to 70/30, v/v, over 12 min), to give the title compound (46 mg, 48%). LC/MS: m/z, 544 (M)⁺, 1.27 min.

### Example 42

### 4-{[3-({[(3′-{[(3S)-3,4-dimethyl-1-piperazinyl]methyl}-6-fluoro-3-biphenylyl)methyl]amino}carbonyl)phenyl]methyl}-1,1-dimethylpiperidinium trifluoroacetate-trifluoroacetic acid (1:1)

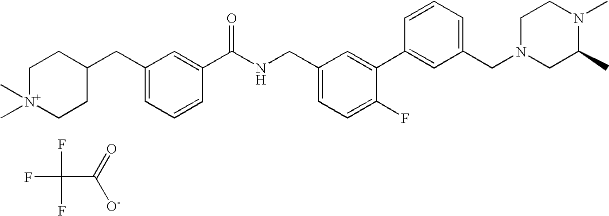

To a solution of 4-{[3-({[(6-fluoro-3′-{[(3S)-3-methyl-1-piperazinyl]methyl}-3-biphenylyl)methyl]amino}carbonyl)phenyl]methyl}-1,1-dimethylpiperidinyl trifluoroacetate (118 mg, 0.19 mmol) in MeOH (5 mL), was added dropwise formaldehyde (37%, 61 mg, 0.76 mmol). After stirring the resulting mixture for 30 min, NaBH₄ (15 mg, 0.38 mmol) was added. The mixture was stirred at room temperature for 16 hours. After removal of the solvent under vacuum, the residue was purified by Gilson reverse phase HPLC, eluting with acetonitrile/water/0.1% TFA (10/90 to 70/30, v/v, over 12 min), to give the title compound (42 mg, 28%). LC/MS: m/z, 557 (M)⁺, 1.38 min.

### Preparation 3

The di-quaternary salt 20 can be prepared from the di-protected intermediate 14 as shown in Scheme 3. The amide 14 sequentially underwent selective deprotection at the piperazine nitrogen followed by reductive amination with formaldehyde to give the tertiary amine 18. Removal of the tert-butyloxy carbonyl group of the piperidine moiety followed by reductive amination with formaldehyde yielded compound 19 which was converted to the di-quaternery salt 20 by reacting with an excess of methyl bromide.

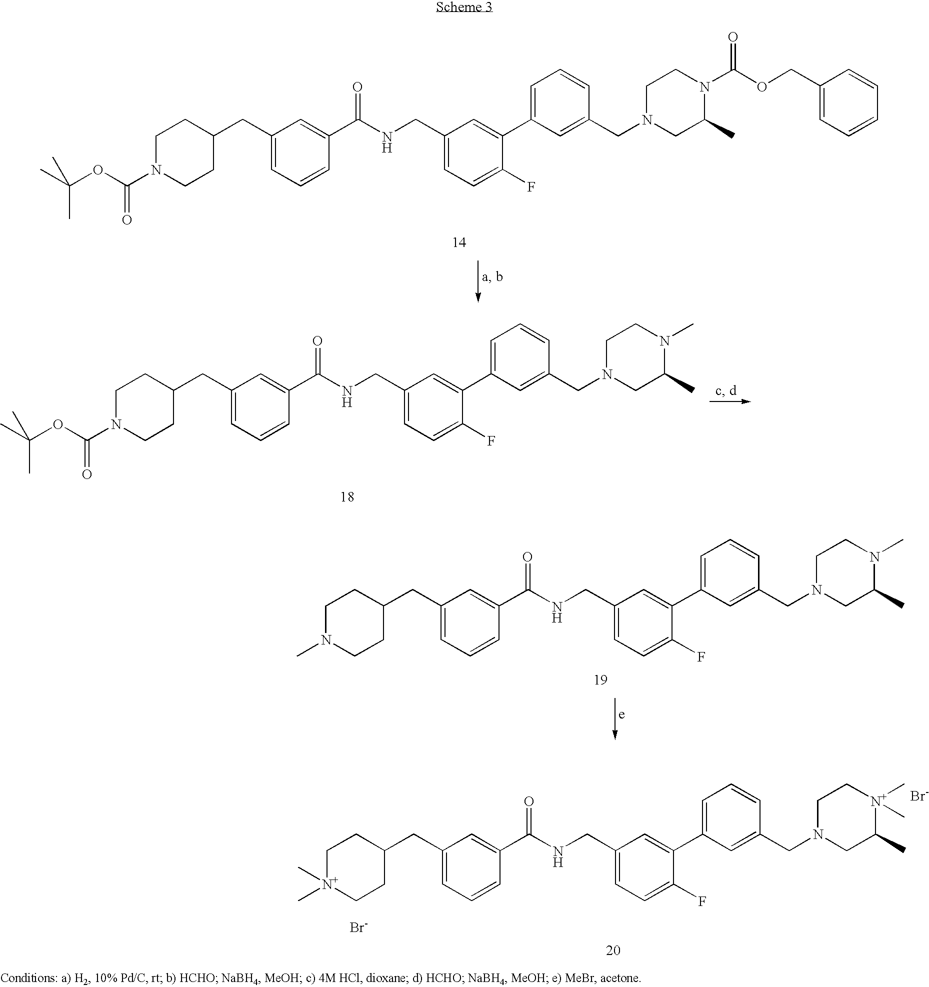

### Intermediate 8: 1,1-Dimethylethyl 4-{[3-({[(6-fluoro-3′-{[(3S)-3-methyl-1-piperazinyl]methyl}-3-biphenylyl)methyl]amino}carbonyl)phenyl]methyl}-1-piperidinecarboxylate

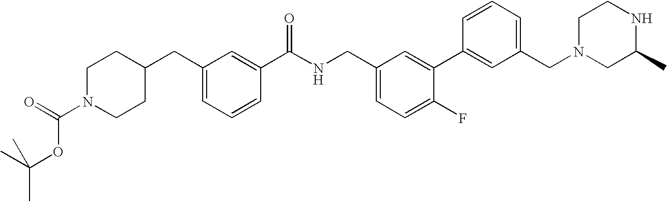

A solution of phenylmethyl (2S)-4-[(5′-{[({3-[(1-{[(1,1-dimethylethyl)oxy]carbonyl}-4-piperidinyl)methyl]phenyl}carbonyl)amino]methyl}-2′-fluoro-3-biphenylyl)methyl]-2-methyl-1-piperazinecarboxylate (1.5 g, 2.0 mmol) and 10% Pd/C (450 mg) in methanol (50 mL) was allowed to react with H₂ at room temperature under atmospheric pressure for 12 hours. The solvent was removed under vacuum. The resulting residue was purified by loading onto 20 g aminopropyl SPE cartridge and eluting sequentially with DCM (3×50 ml), EtOAc (3×50 mL), and MeOH (3×50 mL). The methanol fractions were combined and evaporated to give the title compound as a pale yellow solid (60 mg, 35%). LC/MS: m/z, 615 (M+H), 1.93 min.

### Intermediate 9: 1-dimethylethyl 4-{[3-({[(3′-{[(3S)-3,4-dimethyl-1-piperazinyl]methyl}-6-fluoro-3-biphenylyl)methyl]amino}carbonyl)phenyl]methyl}-1-piperidinecarboxylate

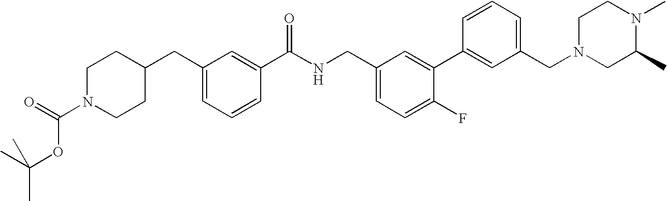

To a solution of 1,1-dimethylethyl 4-([3-({[(6-fluoro-3′-{[(3S)-3-methyl-1-piperazinyl]methyl}-3-biphenylyl)methyl]amino)carbonyl)phenyl]methyl}-1-piperidinecarboxylate (130 mg, 0.21 mmol) in MeOH (5 mL) was added formaldehyde (37% in water, 69 mg, 0.85 mmol). After 30 minutes stirring at rt, sodium borohydride (16 mg, 0.42 mmol) was added. After stirring at rt for a further 3 hours, the solvent was removed to give a residue which was purified by loading onto a 2 g aminopropyl SPE cartridge and eluting sequentially with DCM (3×5 mL), EtOAc. (3×5 mL), and MeOH (3×5 mL). The dichloromethane and ethyl acetate fractions were combined and evaporated to give the title compound (130 mg, 99%). LC/MS: m/z, 629 (M+H), 1.97 min.

### Intermediate 10: N-[(3′-{[(3S)-3,4-dimethyl-1-piperazinyl]methyl}-6-fluoro-3-biphenylyl)methyl]-3-(4-piperidinylmethyl)benzamide

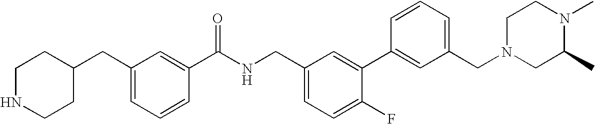

To a solution of 1,1-dimethylethyl 4-{[3-({[(3′-{[(3S)-3,4-dimethyl-1-piperazinyl]methyl}-6-fluoro-3-biphenylyl)methyl]amino}carbonyl)phenyl]methyl}-1-piperidinecarboxylate (250 mg, 0.40 mmol) in 5 mL of 1,4-dioxane was added 4M HCl in 1,4-diozane (5 mL). The mixture was stirred at room temperature for 30 min. After removal of the solvent, the crude was purified by Gilson reverse phase HPLC, eluting with acetonitrile/water/0.1% TFA (10/90 to 70/30, v/v, over 12 min), to give the title compound (200 mg, 95%). LC/MS: m/z, 529 (M+H), 1.27 min.

### Intermediate 11: N-[(3′-{[(3S)-3,4-dimethyl-1-piperazinyl]methyl}-6-fluoro-3-biphenylyl)methyl]-3-[(1-methyl-4-piperidinyl)methyl]benzamide

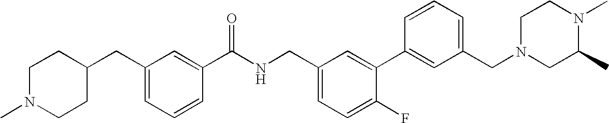

To a solution of N-[(3′-{[(3S)-3,4-dimethyl-1-piperazinyl]methyl}-6-fluoro-3-biphenylyl)methyl]-3-(4-piperidinylmethyl)benzamide (165 mg, 0.313 mmol) in 3 mL of MeOH, was added dropwise formaldehyde (37%, 101 mg, 1.25 mmol). After stirring for 30 minutes, sodium borohydride (24 mg, 0.63 mmol) was added. After stirring of the resulting mixture at rt for 12 hours, the solvent was removed under vacuum and the residue was purified by loading onto a 2 g aminopropyl SPE cartridge and eluting sequentially with DCM (3×5 mL), EtOAc (3×5 mL), and MeOH (3×5 mL). The DCM factions were combined and evaporated to give the title compound as a white solid (60 mg, 35%). LC/MS: m/z, 543 (M+H), 1.18 min.

### Example 43

### (2S)-4-[(5′-{[({3-[(1,1-Dimethyl-4-Piperidiniumyl)methyl]phenyl}carbonyl)amino]methyl}-2′-fluoro-3-biphenylyl)methyl]-1,12-trimethylpiperazin-1-ium dibromide

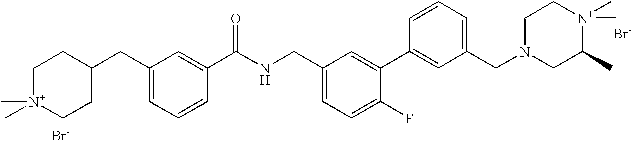

To a solution of N-[(3′-{[(3S)-3,4-dimethyl-1-piperazinyl]methyl}-6-fluoro-3-biphenylyl)methyl]-3-[(1-methyl-4-piperidinyl)methyl]benzamide (15 mg, 0.028 mmol) in acetone (0.2 mL), was added methyl bromide (1 M in t-Butyl ether, 0.3 mL, 0.6 mmol). The mixture was stirred at room temperature for 16 hours then the solvent was removed under vacuum to yield the title compound (20 mg, 100%). LC/MS: m/z, 286 (M/2)⁺, 1.47 min.

### ABBREVIATIONS

BOC tert-butyloxycarbonyl
DCE Dichlroethane
DCM Dichlromethane
DIC 1,3-Dissopropylcarbodiimide
DIPEA Diisopropylethylamine
DME Dimethoxyethane
DMF Dimethylformamide
DMHB 2,6-dimethoxy-4-polystyrenebenzyloxy-benzaldehyde
DMSO Dimethylsulfoxide
ESI Electrospray ionization
HATU O-(7-Azabenzotriazol-1-yl)-N,N,N′,N′-tetramethyluronium hexafluorophosphate
HPLC High pressure liquid chromatography
LC/MS Liquid chromatography/Mass spectrometry
MDAP Mass directed automated preparative
mw microwave
NMP 1-Methyl-2-pyrrolidinone
rt Room temperature
SPE Solid phase extraction
TEA Triethylamine
TFA Trifluoroacetic acid
THF Tetrahydrofuran

### Biological Examples

The inhibitory effects of compounds at the M₃ mAChR of the present invention are determined by the following in vitro and in vivo assays:

### Analysis of Inhibition of Receptor Activation by Calcium Mobilization:

1) 384-well FLIPR Assay

A CHO (chinese hamster ovary) cell line stably expressing the human M3 muscarinic acetylcholine receptor is grown in DMEM plus 10% FBS, 2 mM Glutamine and 200 ug/ml G418. Cells are detached for maintenance and for plating in preparation for assays using either enzymatic or ion chelation methods. The day before the FLIPR (fluorometric imaging plate reader) assay, cells are detached, resuspended, counted, and plated to give 20,000 cells per 384 well in a 50 ul volume. The assay plates are black clear bottom plates, Becton Dickinson catalog number 35 3962. After overnight incubation of plated cells at 37 degrees C. in a tissue culture incubator, the assay is run the next day. To run the assay, media are aspirated, and cells are washed with 1× assay buffer (145 mM NaCl, 2.5 mM KCl, 10 mM glucose, 10 mM HEPES, 1.2 mM MgCl₂, 2.5 mM CaCl₂, 2.5 mM probenecid (pH 7.4.) Cells are then incubated with 50 ul of Fluo-3 dye (4 uM in assay buffer) for 60-90 minutes at 37 degrees C. The calcium-sensitive dye allows cells to exhibit an increase in fluorescence upon response to ligand via release of calcium from intracellular calcium stores. Cells are washed with assay buffer, and then resuspended in 50 ul assay buffer prior to use for experiments. Test compounds and antagonists are added in 25 ul volume, and plates are incubated at 37 degrees C. for 5-30 minutes. A second addition is then made to each well, this time with the agonist challenge, acetylcholine. It is added in 25 ul volume on the FLIPR instrument. Calcium responses are measured by changes in fluorescent units. To measure the activity of inhibitors/antagonists, acetylcholine ligand is added at an EC₈₀ concentration, and the antagonist IC₅₀ can then be determined using dose response dilution curves. The control antagonist used with M3 is atropine.

2) 96-well FLIPR Assay

Stimulation of mAChRs expressed on CHO cells were analyzed by monitoring receptor-activated calcium mobilization as previously described. CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl₂, 1.1 mM MgCl₂, 11 mM glucose, 20 mM HEPES (pH 7.4)). 50 μl of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels. The emitted fluorescence from all 96 wells is measured simultaneously using a cooled CCD camera. Data points are collected every second. This data was then plotting and analyzed using GraphPad PRISM software.

### Methacholine-Induced Bronchoconstriction

Airway responsiveness to methacholine was determined in awake, unrestrained BalbC mice (n=6 each group). Barometric plethysmography was used to measure enhanced pause (Penh), a unitless measure that has been shown to correlate with the changes in airway resistance that occur during bronchial challenge with methacholine. Mice were pretreated with 50 μl of compound (0.003-10 μg/mouse) in 50 μl of vehicle (10% DMSO) intranasally, and were then placed in the plethysmography chamber. Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software. This experiment allows the determination of duration of activity of the administered compound.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis.

### Formulation-Administration

Accordingly, the present invention further provides a pharmaceutical formulation comprising a compound of formula (I), or a pharmaceutically acceptable salt, solvate, or physiologically functional derivative (e.g., salts and esters) thereof, and a pharmaceutically acceptable carrier or excipient, and optionally one or more other therapeutic ingredients.

Hereinafter, the term “active ingredient” means a compound of formula (I), or a pharmaceutically acceptable salt, solvate, or physiologically functional derivative thereof.

Compounds of formula (I) will be administered via inhalation via the mouth or nose.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di- or poly-saccharides (e.g., lactose or starch), organic or inorganic salts (e.g., calcium chloride, calcium phosphate or sodium chloride), polyalcohols (e.g., mannitol), or mixtures thereof, alternatively with one or more additional materials, such additives included in the blend formulation to improve chemical and/or physical stability or performance of the formulation, as discussed below, or mixtures thereof. Use of lactose is preferred. Each capsule or cartridge may generally contain between 20 μg-10 mg of the compound of formula (I) optionally in combination with another therapeutically active ingredient. Alternatively, the compound of the invention may be presented without excipients, or may be formed into particles comprising the compound, optionally other therapeutically active materials, and excipient materials, such as by co-precipitation or coating.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant as an inhaler having a reservoir form pack suitable for comprising multiple (un-metered doses) of medicament in dry powder form and including means for metering medicament dose from the reservoir to a delivery position. The metering means may for example comprise a metering cup or perforated plate, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disk-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants eg oleic acid or lecithin and cosolvents eg ethanol. Pressurized formulations will generally be retained in a canister (eg an aluminium canister) closed with a valve (eg a metering valve) and fitted into an actuator provided with a mouthpiece.

Medicaments for administration by inhalation desirably have a controlled particle size. The optimum aerodynamic particle size for inhalation into the bronchial system for localized delivery to the lung is usually 1-10 μm, preferably 2-5 μm. The optimum aerodynamic particle size for inhalation into the alveolar region for achieving systemic delivery to the lung is approximately 0.5-3 μm, preferably 1-3 μm. Particles having an aerodynamic size above 20 μm are generally too large when inhaled to reach the small airways. Average aerodynamic particle size of a formulation may measured by, for example cascade impaction. Average geometric particle size may be measured, for example by laser diffraction, optical means.

To achieve a desired particle size, the particles of the active ingredient as produced may be size reduced by conventional means eg by controlled crystallization, micronisation or nanomilling. The desired fraction may be separated out by air classification. Alternatively, particles of the desired size may be directly produced, for example by spray drying, controlling the spray drying parameters to generate particles of the desired size range. Preferably, the particles will be crystalline, although amorphous material may also be employed where desirable. When an excipient such as lactose is employed, generally, the particle size of the excipient will be much greater than the inhaled medicament within the present invention, such that the “coarse” carrier is non-respirable. When the excipient is lactose it will typically be present as milled lactose, wherein not more than 85% of lactose particles will have a MMD of 60-90 μm and not less than 15% will have a MMD of less than 15 μm. Additive materials in a dry powder blend in addition to the carrier may be either respirable, i.e., aerodynamically less than 10 microns, or non-respirable, i.e., aerodynamically greater than 10 microns.

Suitable additive materials which may be employed include amino acids, such as leucine; water soluble or water insoluble, natural or synthetic surfactants, such as lecithin (e.g., soya lecithin) and solid state fatty acids (e.g., lauric, palmitic, and stearic acids) and derivatives thereof (such as salts and esters); phosphatidylcholines; sugar esters. Additive materials may also include colorants, taste masking agents (e.g., saccharine), anti-static-agents, lubricants (see, for example, Published PCT Patent Appl. No. WO 87/905213, the teachings of which are incorporated by reference herein), chemical stabilizers, buffers, preservatives, absorption enhancers, and other materials known to those of ordinary skill.

Sustained release coating materials (e.g., stearic acid or polymers, e.g. polyvinyl pyrolidone, polylactic acid) may also be employed on active material or active material containing particles (see, for example, Patent Nos. U.S. Pat. No. 3,634,582, GB 1,230,087, GB 1,381,872, the teachings of which are incorporated by reference herein).

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Preferred unit dosage formulations are those containing an effective dose, as herein before recited, or an appropriate fraction thereof, of the active ingredient.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

1. A compound of formula I as indicated below: 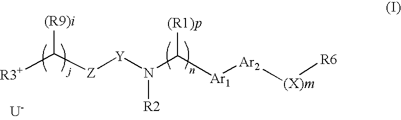
wherein
Ar1 and Ar2, are independently, selected from the group consisting of optionally substituted phenyl and optionally substituted monocyclic heteroaryl;
R6 is NR₇R₈, or an optionally substituted saturated or partially unsaturated 4-10 membered ring system in which one or more rings contain one or more secondary nitrogens, tertiary nitrogens, or quaternary ammonium nitrogens, and optionally contain one or more O, or S;
X is C(R1)p, or C(O); wherein, when X is C(R1)p, m is an integer from 0 to 3; when X is C(O), m is 1;
p is an integer from 0 to 2;
i is an integer from 0 to 2;
n is an integer from 0 to 3;
j is an integer from 0 to 3;
Y is C(O), S(O)q, HNC(O), or OC(O); wherein, q is 1 or 2;
R1, R2, and R9 are independently selected from the group consisting of hydrogen, optionally substituted C₁-C₁₀ alkyl, optionally substituted C₃-C₁₀ cycloalkyl, optionally substituted C₃-C₁₀ cycloalkyl alkyl, optionally substituted heterocylic, optionally substituted heterocyclicalkyl, optionally substituted alkenyl, optionally substituted aryl, optionally substituted aryl alkyl, optionally substituted heteroaryl, and optionally substituted heteroaryl alkyl;
Z is selected from the group consisting of optionally substituted aryl, optionally substituted heteroaryl, optionally substituted alkenyl, optionally substituted C₁-C₁₀ alkyl, optionally substituted C₃-C₁₀ cycloalkyl, optionally substituted C₃-C₁₀ cycloalkyl alkyl, optionally substituted aryl alkyl, and optionally substituted heteroaryl alkyl;
R₃⁺ is N⁺R₄R₅R₁₀, or an optionally substituted saturated or partially unsaturated 4-10 membered ring system in which one or more rings contain one or more quaternary ammonium nitrogens, and optionally contain one or more secondary or tertiary nitrogens, O, or S;
U⁻ is a pharmaceutically acceptable counter ion, selected from the group consisting of I⁻, Br⁻, Cl⁻, F⁻, CF3COO⁻, mesylate, and tosylate;
R₄, R₅, and R₁₀, are independently, selected from the group consisting of optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or any two or three of R₄, R₅, and R₁₀, together with the nitrogen to which they are attached, form a 5 to 10 membered ring system which may optionally comprise an additional heteroatom selected from O, N and S;
R₇ and R₈, are independently, selected from the group consisting of hydrogen, optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or R₇ and R₈, together with the nitrogen to which they are attached, form a 5 to 7 member ring which may optionally comprise an additional heteroatom selected from O, N and S;
or any other pharmaceutically acceptable salt thereof.

2. A compound according to claim 1 wherein
Ar1 and Ar2, are independently, selected from the group consisting of optionally substituted phenyl and optionally substituted monocyclic heteroaryl;
R₆ is an optionally substituted saturated or partially unsaturated 4-10 membered ring system in which one or more rings contain one or more secondary nitrogens, tertiary nitrogens, or quaternary ammonium nitrogens;
X is C(R₁)p;
p is 2;
m is an integer from 0 to 3;
i is 2;
n is an integer from 1 to 3;
j is an integer from 0 to 3;
Y is C(O), or S(O)q; wherein, q is 1 or 2;
R1 is hydrogen;
R9 is hydrogen;
R2 is selected from the group consisting of hydrogen, optionally substituted C₁-C₁₀ alkyl, optionally substituted alkenyl, optionally substituted C₃-C₁₀ cycloalkyl, optionally substituted C₃-C₁₀ cycloalkyl alkyl, optionally substituted heterocylic, optionally substituted heterocyclicalkyl, optionally substituted aryl, optionally substituted aryl alkyl, optionally substituted heteroaryl, and optionally substituted heteroaryl alkyl;
Z is selected from the group consisting of optionally substituted aryl, optionally substituted heteroaryl, optionally substituted aryl alkyl, and optionally substituted heteroaryl alkyl;
R₃⁺ is N⁺R₄R₅R₁₀, or an optionally substituted saturated or partially unsaturated 4-10 membered ring system in which one or more rings contain one or more quaternary ammonium nitrogens, and optionally contain one or more secondary or tertiary nitrogens, O, or S;
U⁻ is a pharmaceutically acceptable counter ion, selected from the group consisting of I⁻, Br⁻, Cl⁻, F⁻, CF3COO⁻, mesylate, and tosylate;
R₄, R₅, and R₁₀, are independently, selected from the group consisting of optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or any two or three of R₄, R₅, and R₁₀, together with the nitrogen to which they are attached, form a 5 to 10 membered ring system which may optionally comprise an additional heteroatom selected from O, N and S;
or any other pharmaceutically acceptable salt thereof.

3. A compound according to claim 1 wherein
Ar1 and Ar2, are independently, selected from the group consisting of optionally substituted phenyl and optionally substituted monocyclic heteroaryl;
R₆ is an optionally substituted saturated or partially unsaturated 5-8 membered ring system in which one or more rings contain one or more secondary or tertiary nitrogens;
X is C(R1)p;
p is 2;
m is 1;
i is 2;
n is 1;
j is 1, or 0;
Y is C(O), or S(O)q; wherein, q is 1 or 2;
R1 is hydrogen;
R9 is hydrogen;
R2 is selected from the group consisting of hydrogen, optionally substituted C₁-C₁₀ alkyl, optionally substituted alkenyl, optionally substituted C₃-C₁₀ cycloalkyl, optionally substituted C₃-C₁₀ cycloalkyl alkyl, optionally substituted heterocylic, optionally substituted heterocyclicalkyl, optionally substituted aryl alkyl, and optionally substituted heteroaryl alkyl;
Z is selected from the group consisting of optionally substituted phenyl, optionally substituted heteroaryl, optionally substituted phenyl alkyl, and optionally substituted heteroaryl alkyl;
R₃⁺ is N⁺R₄R₅R₁₀, or an optionally substituted saturated or partially unsaturated 4-10 membered ring system in which one or more rings contain one or more quaternary ammonium nitrogens, and optionally contain one or more secondary or tertiary nitrogens, O, or S;
U⁻ is a pharmaceutically acceptable counter ion, selected from the group consisting of I⁻, Br⁻, Cl⁻, F⁻, CF3COO⁻, mesylate, and tosylate;
R₄, R₅, and R₁₀, are independently, selected from the group consisting of optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or any two or three of R₄, R₅, and R₁₀, together with the nitrogen to which they are attached, form a 5 to 10 membered ring system which may optionally comprise an additional heteroatom selected from O, N and S;
or any other pharmaceutically acceptable salt thereof.

4. A compound according to claim 1 selected from the group consisting of:
4-{[3-({[(6-fluoro-3′-{[(3S)-3-methyl-1-piperazinyl]methyl}-3-biphenylyl)methyl]amino}carbonyl)phenyl]methyl}-1,1-dimethylpiperazin-1-ium trifluoroacetate trifluoroacetic acid (1:3);
4-({3-[({[6-fluoro-3′-(1-piperazinylmethyl)-3-biphenylyl]methyl}amino)carbonyl]phenyl}methyl)-1,1-dimethylpiperazin-1-ium trifluoroacetate trifluoroacetic acid (1:3);
4-[(3-{[({3′-[(1S,4S)-2,5-diazabicyclo[2.2.1]hept-2-ylmethyl]-6-fluoro-3-biphenylyl}methyl)amino]carbonyl}phenyl)methyl]-1,1-dimethylpiperazin-1-ium trifluoroacetate trifluoroacetic acid (1:3);
1,1-dimethyl-4-({3-[({[6-(methyloxy)-3′-(1-piperazinylmethyl)-3-biphenylyl]methyl}amino)carbonyl]phenyl}methyl)piperazin-1-ium trifluoroacetate trifluoroacetic acid (1:3);
1,1-dimethyl-4-{[3-({[(6-(methyloxy)-3′-{[(3S)-3-methyl-1-piperazinyl]methyl}-3-biphenylyl)methyl]amino}carbonyl)phenyl]methyl}piperazin-1-ium trifluoroacetate trifluoroacetic acid (1:3);
4-({3-[({[3′-[(1S,4S)-2,5-diazabicyclo[2.2.1]hept-2-ylmethyl]-6-(methyloxy)-3-biphenylyl]methyl}amino)carbonyl]phenyl}methyl)-1,1-dimethylpiperazin-1-ium trifluoroacetate trifluoroacetic acid (1:3);
(1S,4S)-2-{[3-({[(6-fluoro-3′-{[(3S)-3-methyl-1-piperazinyl]methyl}-3-biphenylyl)methyl]amino}carbonyl)phenyl]methyl}-2-methyl-5-aza-2-azoniabicyclo[2.2.1]heptane trifluoroacetate trifluoroacetic acid (1:3);
1-{[3-({[(6-fluoro-3′-{[(3S)-3-methyl-1-piperazinyl]methyl}-3-biphenylyl)methyl]amino}carbonyl)phenyl]methyl}-1-methylpiperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1,1-dimethylhexahydro-1H-1,4-diazepin-1-ium trifluoroacetate trifluoroacetic acid (1:3);
1-{[3-({[(6-fluoro-3′-{[(3S)-3-methyl-1-piperazinyl]methyl}-3-biphenylyl)methyl]amino}carbonyl)phenyl]methyl}-1-methylhexahydro-1H-1,4-diazepin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-(3-hydroxypropyl)-1-methylpiperazin-1-ium trifluoroacetate trifluoroacetic acid (1:3);
(2R,5S)-4-{[3-({[(6-fluoro-3′-{[(3S)-3-methyl-1-piperazinyl]methyl}-3-biphenylyl)methyl]amino}carbonyl)phenyl]methyl}-1,1,2,5-tetramethylpiperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-[3-(methyloxy)propyl]piperazin-1-ium trifluoroacetate trifluoroacetic acid (1:3);
3-{[3-({[(6-fluoro-3′-{[(3S)-3-methyl-1-piperazinyl]methyl}-3-biphenylyl)methyl]amino}carbonyl)phenyl]methyl}-3-aza-6-azoniaspiro[5.6]dodecane trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-[(2E)-3-phenyl-2-propen-1-yl]piperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-propylpiperazin-1-ium trifluoroacetate trifluoroacetic acid (1:3);
2-(aminocarbonyl)-4-{[3-({[(6-fluoro-3′-{[(3S)-3-methyl-1-piperazinyl]methyl}-3-biphenylyl)methyl]amino}carbonyl)phenyl]methyl}-1,1-dimethylpiperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-(phenylmethyl)piperazin-1-ium trifluoroacetate trifluoroacetic acid (1:3);
3-{[3-({[(6-fluoro-3′-{[(3S)-3-methyl-1-piperazinyl]methyl}-3-biphenylyl)methyl]amino}carbonyl)phenyl]methyl}-3-aza-6-azoniaspiro[5.5]undecane trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-[2-(phenyloxy)ethyl]piperazin-1-ium trifluoroacetate trifluoroacetic acid (1:3);
1-ethyl-4-{[3-({[(6-fluoro-3′-{[(3S)-3-methyl-1-piperazinyl]methyl}-3-biphenylyl)methyl]amino}carbonyl)phenyl]methyl}-1-methylpiperazin-1-ium trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1-methyl-1-(2-propen-1-yl)piperazin-1-ium trifluoroacetate trifluoroacetic acid (1:3);
(1S,4S)-5-{[3-({[(6-fluoro-3′-{[(3S)-3-methyl-1-piperazinyl]methyl}-3-biphenylyl)methyl]amino}carbonyl)phenyl]methyl}-2,2-dimethyl-5-aza-2-azoniabicyclo[2.2.1]heptane trifluoroacetate trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1,1,2-trimethylpiperazin-1-ium trifluoroacetate trifluoroacetic acid (1:3);
1-(3-cyanopropyl)-4-{[3-({[(6-fluoro-3′-{[(3S)-3-methyl-1-piperazinyl]methyl}-3-biphenylyl)methyl]amino}carbonyl)phenyl]methyl}-1-methylpiperazin-1-ium trifluoroacetate trifluoroacetic acid (1:3);
1-{[3-({[(6-fluoro-3′-{[(3S)-3-methyl-1-piperazinyl]methyl}-3-biphenylyl)methyl]amino}carbonyl)phenyl]methyl}-1,4-dimethylpiperidinium trifluoroacetate trifluoroacetic acid (1:2);
4-[2-(4-{[3-({[(6-fluoro-3′-{[(3S)-3-methyl-1-piperazinyl]methyl}-3-biphenylyl)methyl]amino}carbonyl)phenyl]methyl}-1-piperazinyl)ethyl]-4-methylmorpholin-4-ium trifluoroacetate trifluoroacetic acid (1:4);
1-{[3-({[(6-fluoro-3′-{[(3S)-3-methyl-1-piperazinyl]methyl}-3-biphenylyl)methyl]amino}carbonyl)phenyl]methyl}-4-formyl-1-methylpiperazin-1-ium trifluoroacetate trifluoroacetic acid (1:3);
1-{[3-({[(6-fluoro-3′-{[(3S)-3-methyl-1-piperazinyl]methyl}-3-biphenylyl)methyl]amino}carbonyl)phenyl]methyl}-1-methylpiperidinium trifluoroacetate trifluoroacetic acid (1:2);
4-{[3-({[(6-fluoro-3′-{[(3S)-3-methyl-1-piperazinyl]methyl}-3-biphenylyl)methyl]amino}carbonyl)phenyl]methyl}-4-methylmorpholin-4-ium trifluoroacetate trifluoroacetic acid (1:2);
[3-({[(6-fluoro-3′-{[(3S)-3-methyl-1-piperazinyl]methyl}-3-biphenylyl)methyl]amino}carbonyl)phenyl]-N,N,N-trimethylmethanaminium trifluoroacetate trifluoroacetic acid (1:2);
N-ethyl-N-{[3-({[(6-fluoro-3′-{[(3S)-3-methyl-1-piperazinyl]methyl}-3-biphenylyl)methyl]amino}carbonyl)phenyl]methyl}-N-methylethanaminium trifluoroacetate trifluoroacetic acid (1:2);
1-[2-(4-{[3-({[(6-fluoro-3′-{[(3S)-3-methyl-1-piperazinyl]methyl}-3-biphenylyl)methyl]amino}carbonyl)phenyl]methyl}-1-piperazinyl)ethyl]-1-methylpyrrolidinium iodide trifluoroacetic acid (1:3);
3-[{[3-({[(6-fluoro-3′-{[(3S)-3-methyl-1-piperazinyl]methyl}-3-biphenylyl)methyl]amino}carbonyl)phenyl]methyl}(methyl)amino]-1,1-dimethylpyrrolidinium iodide trifluoroacetic acid (1:3);
2-[{[3-({[(6-fluoro-3′-{[(3S)-3-methyl-1-piperazinyl]methyl}-3-biphenylyl)methyl]amino}carbonyl)phenyl]methyl}(methyl)amino]-N,N,N-trimethylethanaminium iodide trifluoroacetic acid (1:3);
4-(4-{[3-({[(6-fluoro-3′-{[(3S)-3-methyl-1-piperazinyl]methyl}-3-biphenylyl)methyl]amino}carbonyl)phenyl]methyl}-1-piperazinyl)-1,1-dimethylpiperidinium iodide trifluoroacetic acid (1:4);
2-(1-{[3-({[(6-fluoro-3′-{[(3S)-3-methyl-1-piperazinyl]methyl}-3-biphenylyl)methyl]amino}carbonyl)phenyl]methyl}-4-piperidinyl)-N,N,N-trimethylethanaminium iodide trifluoroacetic acid (1:3);
7-{[3-({[(6-fluoro-3′-{[(3S)-3-methyl-1-piperazinyl]methyl}-3-biphenylyl)methyl]amino}carbonyl)phenyl]methyl}-1,1-dimethyl-7-aza-1-azoniaspiro[4.4]nonane iodide trifluoroacetic acid (1:3);
(1-{[3-({[(6-fluoro-3′-{[(3S)-3-methyl-1-piperazinyl]methyl}-3-biphenylyl)methyl]amino}carbonyl)phenyl]methyl}-2-piperidinyl)-N,N,N-trimethylmethanaminium iodide trifluoroacetic acid (1:3);
3-[{[3-({[(6-fluoro-3′-{[(3S)-3-methyl-1-piperazinyl]methyl}-3-biphenylyl)methyl]amino}carbonyl)phenyl]methyl}(methyl)amino]-N,N,N-trimethyl-1-propanaminium iodide trifluoroacetic acid (1:3);
4-{[3-({[(6-fluoro-3′-{[(3S)-3-methyl-1-piperazinyl]methyl}-3-biphenylyl)methyl]amino}carbonyl)phenyl]methyl}-1,1-dimethylpiperidinium trifluoroacetate trifluoroacetic acid (1:1);
(2S)-4-[(5′-{[({3-[(1,1-dimethyl-4-piperidiniumyl)methyl]phenyl}carbonyl)amino]methyl}-2′-fluoro-3-biphenylyl)methyl]-1,1,2-trimethylpiperazin-1-ium dibromide; and
4-{[3-({[(3′-{[(3S)-3,4-dimethyl-1-piperazinyl]methyl}-6-fluoro-3-biphenylyl)methyl]amino}carbonyl)phenyl]methyl}-1,1-dimethylpiperidinium trifluoroacetate trifluoroacetic acid (1:1);
or any other pharmaceutically acceptable salt thereof.

5. A pharmaceutical composition for the treatment of muscarinic acetylcholine receptor mediated diseases comprising a compound according to claim 1 and a pharmaceutically acceptable carrier thereof.

6. A method of inhibiting the binding of acetylcholine to its receptors in a mammal in need thereof comprising administering a safe and effective amount of a compound according to claim 1.

7. A method of treating a muscarinic acetylcholine receptor mediated disease, wherein acetylcholine binds to said receptor, comprising administering a safe and effective amount of a compound according to claim 1.

8. A method according to claim 7 wherein the disease is selected from the group consisting of chronic obstructive lung disease, chronic bronchitis, asthma, chronic respiratory obstruction, pulmonary fibrosis, pulmonary emphysema and allergic rhinitis.

9. A method according to claim 8 wherein administration is via inhalation via the mouth or nose.

10. A method according to claim 9 wherein administration is via a medicament dispenser selected from a reservoir dry powder inhaler, a multi-dose dry powder inhaler or a metered dose inhaler.

11. A method according to claim 10 wherein the compound is administered to a human and has a duration of action of 12 hours or more.

12. A method according to claim 11 wherein the compound has a duration of action of 24 hours or more.

13. A method according to claim 12 wherein the compound has a duration of action of 36 hours or more.
